(19) United States
(12) Patent Application Publication
(10) Pub. No.: US 2020/0282008 A1
(43) Pub. Date: Sep. 10, 2020

(54) COMPOSITIONS AND METHODS FOR LONG TERM RELEASE OF GONADOTROPIN-RELEASING HORMONE (GnRH) ANTAGONISTS

(71) Applicant: VERU INC., Miami, FL (US)

(72) Inventors: Ravi KACKER, LEXINGTON, MA (US); Mitchell S. STEINER, GERMANTOWN, TN (US); Jui-Chen LIN, San Diego, CA (US); Andrew Michael CERRO, SAN DIEGO, CA (US); Christopher A. RHODES, SAN DIEGO, CA (US)

(21) Appl. No.: 16/880,309

(22) Filed: May 21, 2020

Related U.S. Application Data
(63) Continuation-in-part of application No. 15/974,461, filed on May 8, 2018.
Application No. 15/974,461 is a continuation-in-part of application No. 15/885,464, filed on Jan. 31, 2018.
(60) Provisional application No. 62/452,788, filed on Jan. 31, 2017.

(51) Int. Cl. A61K 38/09; A61K 9/06; A61K 47/34; A61P 35/00; A61P 5/26; A61K 9/00

(52) U.S. Cl. CPC A61K 38/09; A61K 9/06; A61K 9/0024; A61P 35/00; A61P 5/26; A61K 47/34

(57) ABSTRACT

The invention provides compositions and methods for long term release of gonadotropin-releasing hormone (GnRH) antagonists and uses thereof. Specifically, the invention provides polymer compositions and methods for controlled release of GnRH antagonists.

### CROSS-REFERENCE TO RELATED APPLICATIONS

This application is a continuation-in-part application of U.S. application Ser. No. 15/974,461, filed May 8, 2018, which is a continuation-in-part application of U.S. application Ser. No. 15/885,464, filed Jan. 31, 2018, which claims priority to and the benefit of U.S. Provisional Application 62/452,788, filed Jan. 31, 2017, incorporated by reference herein.

### FIELD OF THE INVENTION

The invention relates to compositions and methods for long term release of gonadotropin-releasing hormone (GnRH) antagonists and uses thereof. Specifically, the invention relates to polymer-based compositions and methods for controlled release of GnRH antagonists.

### BACKGROUND OF THE INVENTION

The hypothalamic hormone, gonadotropin-releasing hormone (GnRH) (also known as luteinizing hormone releasing hormone (LHRH)), controls the secretion of the gonadotropins, luteinizing hormone (LH) and follicle stimulating hormone (FSH) from the anterior pituitary gland. Analogues of GnRH are currently used to treat many medical conditions that require manipulation of the production of the sex hormones, testosterone and estrogen. Schally et al. (Schally 1971) isolated, identified the amino acid sequence, and synthesized the peptide hormone GnRH. Deletion or replacement of different amino acids of GnRH peptide has resulted in the discovery of distinct GnRH agonist analogues that demonstrate greater relative potency for the secretion of LH and FSH. A paradoxical clinical effect occurs when agonistic analogues are used continuously such that after the chronic, and relatively long period (2-3 weeks) of stimulation of the secretion of LH and FSH, there is actually an inhibition of LH and/or FSH release and consequent suppression of sex steroid production (testosterone and estrogen). (Reissmann 2000). In certain medical conditions, however, an immediate and dose-dependent suppression of LH and FSH is desired. Over 20 years ago, Schally and Revier synthesized the Pt generation analogues of GnRH antagonist analogues which were too lipophilic and induced histamine release. (Schmidt 1984; Hahn 1985). The 2^{nd} generation GnRH antagonist analogues were made by incorporating further amino acid substitutions (Bajusz 1988; Rivier 1993) that resulted in potentially safer and more effective decapeptide analogues. Examples of newer generation GnRH antagonist analogues include degarelix, ganirelix, ozarelix, cetrorelix, taverelix, antarelix, and iturelix.

Clinical development and medical applications of these GnRH antagonist analogues have been either successful or attempted for controlled ovarian stimulation for assisted reproductive techniques, uterine myoma, ovarian cancer, benign prostatic hyperplasia, and prostate cancer. In certain diseases and conditions, the major limitation for successful application of the GnRH antagonist analogue has been having only a short acting formulation where longer acting depot formulations would be more clinically advantageous for optimal drug compliance.

Accordingly, there exists a need for very long acting controlled or extended release formulations of a GnRH antagonist (also called a LHRH antagonist).

### SUMMARY OF THE INVENTION

In one aspect, the invention relates to a long-term drug release composition comprising: a therapeutically effective amount of a GnRH antagonist in combination with a multi-block copolymer, wherein the composition releases the GnRH antagonist for a duration of at least six months.

In another aspect, the invention relates to a flowable composition, the composition comprising: (a) a multi-block copolymer; (b) a biocompatible polar aprotic solvent, wherein the biocompatible polar aprotic solvent is miscible to dispersible in aqueous medium or body fluid; and (c) a therapeutically effective amount of a GnRH antagonist, wherein the flowable composition releases the GnRH antagonist for a duration of at least four months. In exemplary embodiments of the compositions, the GnRH antagonist is cetrorelix, degarelix, ganirelix, ozarelix, taverelix, antarelix, or iturelix. In various embodiments, the multi-block copolymer is polyglycolide (PLG), polylactide (PLA), or poly-lactic co-glycolic acid (PLGA).

In another aspect, the invention relates to a composition for a long-term release of cetrorelix, the composition comprising a multi-block copolymer, a solvent, and a therapeutically effective amount of cetrorelix, and the release of cetrorelix duration is at least six months.

In another aspect, the invention relates to a method to extend the release of cetrorelix in a subject for a duration of at least six months. The method comprising administering to the subject a composition comprising cetrorelix and a multi-block copolymer. Wherein the multi-block copolymer comprises poly-lactic co-glycolic acid (PLGA) in a lactide:glycolide molar ratio between 1:1, 1:0, and 0:1. Wherein cetrorelix is present in an amount of 5%-90% of the weight of the composition, and the multi-block copolymer is present in an amount of 10%-50% of the weight of the composition. In other embodiments, the composition comprises cetrorelix and a multi-block copolymer, the multi-block copolymer comprising poly-lactic co-glycolic acid (PLGA) in a lactide:glycolide molar ratio of 3:1. In certain embodiments, the composition comprises cetrorelix and a multi-block copolymer. The multi-block copolymer comprising poly-lactic co-glycolic acid (PLGA) in a lactide:glycolide molar ratio of 5.67:15.

In another aspect, the invention relates to a method to maintain a therapeutic level of cetrorelix in a subject for a duration of at least six months. The method comprising administering to the subject a composition comprising cetrorelix and a multi-block copolymer. The multi-block copolymer comprising poly-lactic co-glycolic acid (PLGA) in a lactide:glycolide molar ratio between 1:1, 1:0 and 0:1. Wherein cetrorelix is present in an amount of 5%-90% by the weight of the composition, and the multi-block copolymer is present in an amount of 10%-50% by the weight of the composition.

In another aspect, the invention relates to a composition comprising: a therapeutically effective amount of a GnRH antagonist in combination with a multi-block copolymer. Wherein the multi-block copolymer comprises polyethyleneglycol(PEG)-PLGA-PEG, poly(3-hydroxybutyrate), PCL, poly(glycolide) (PLG), poly(lactide) (PLA), poly-lactic co-glycolic acid (PLGA) or a combination thereof. Wherein the composition achieves a therapeutic effect within 24 hours and maintains therapeutic effect for at least 180 days. In an exemplary embodiment, GnRH antagonist is cetrorelix, degarelix, ganirelix, ozarelix, taverelix, antarelix, or iturelix.

In another aspect, the invention relates to a composition comprising: a therapeutically effective amount of a GnRH antagonist in combination with a multi-block copolymer. Wherein the multi-block copolymer comprises randomly or non-alternatingly arranged hydrolysable segments. Wherein each segment comprises pre-polymer A or pre-polymer B, and wherein the segments are operably linked to each other by a multifunctional chain extender. Wherein the composition achieves a therapeutic effect within 24 hours and maintains therapeutic effect for at least 180 days.

In another aspect, the invention relates to a method for treating a disease or condition associated with gonadotropin-releasing hormone (GnRH). The method comprising administering to a subject a composition of any of the above, or below, embodiments, thereby treating the disease in the subject. Wherein the composition achieves a therapeutic effect within 24 hours and maintains therapeutic effect for at least 180 days.

In another aspect, the invention relates to a composition, the composition comprising: a therapeutically effective amount of a GnRH antagonist in combination with a non-PLGA multi-block copolymer. In one aspect the multi-block copolymer is biodegradable. In another aspect the multi-block copolymer is a thermoplastic polyester that is substantially insoluble/stable in an aqueous medium or body fluid.

In one aspect the multi-block copolymer is at least one of polyethylene glycol (PEG), PLG, PLA, polybutylene terephthalate (PBT), poly(epsilon-caprolactone) (PCL), dioxanone, butanediisocyanate, butanediol, polyoxyethylene, polypropylene, polyoxypropylene, polystyrene, poly methyl methacrylate. Wherein the composition releases the GnRH antagonist for a long term.

In another aspect, the invention relates to a flowable composition, the composition comprising: (a) a biodegradable thermoplastic polyester that is at least substantially insoluble/stable in aqueous medium or body fluid; (b) a biocompatible solvent, wherein the biocompatible solvent is miscible to dispersible in aqueous medium or body fluid; and (c) a therapeutically effective amount of a GnRH antagonist.

In another aspect, the invention relates to a flowable composition, the composition comprising: (a) a biodegradable thermoplastic polyester that is at least substantially insoluble/stable in aqueous medium or body fluid; (b) a biocompatible polar aprotic solvent, wherein the biocompatible polar aprotic solvent is miscible to dispersible in aqueous medium or body fluid; and (c) a therapeutically effective amount of a GnRH antagonist.

In an exemplary embodiment, the thermoplastic polyester is a polylactide, a polyglycolide, a polycaprolactone, a copolymer thereof, a terpolymer thereof, or any combination thereof.

In another exemplary embodiment, the solvent is N-methyl-2-pyrrolidone, 2-pyrrolidone, N,N-dimethylformamide, dimethyl sulfoxide, propylene carbonate, caprolactam, triacetin, benzyl benzoate, propylene glycol, or any combination thereof. In a particular embodiment, the flowable composition of the invention comprises a flowable delivery system such as an Atrigel® system comprising a copolymer, a water-soluble organic solvent, and a bioactive agent, for example, a GnRH antagonist.

In another aspect, the invention relates to a composition, the composition comprising: a therapeutically effective amount of a GnRH antagonist in combination with a multi-block copolymer, wherein the multi-block copolymer comprises polyethyleneglycol(PEG)-PLGA-PEG, poly(3-hydroxybutyrate), PCL, PLG, PLA, or a combination thereof.

In another aspect, the invention relates to a composition, the composition comprising: a therapeutically effective amount of a GnRH antagonist in combination with a multi-block copolymer, wherein the multi-block copolymer comprises randomly or non-alternatingly arranged hydrolysable segments, wherein each segment comprises pre-polymer A or pre-polymer B, and wherein the segments are operably linked to each other by a multifunctional chain extender. In one aspect the repeating units of the multi-block copolymer can be the same subunit. In an exemplary embodiment, the multi-block copolymer is a phase separated multiblock copolymer, comprising: one or more segments of a linear soft biodegradable pre-polymer A having a glass transition temperature (T_{g}) lower than 37° C.; and one or more segments of a linear hard biodegradable pre-polymer B having a melting point temperature (Tₘ) of 40-100° C.

In another aspect, the invention relates to the use of salt bridges or cyclization of the active agent either as a primary drug delivery technique or in combination with another drug delivery vehicle using compounds that include, but are not limited to, lanthionine, dicarba, hydrazine, or lactam bridges.

In another aspect, the invention relates to the use of micronization or stabilizing adjuvants for a long-term delivery of a GnRH antagonist.

In another aspect, the invention relates to the use of a solid-in-oil-in-water (S/O/W), a water-in-oil-in water (W/O/W), or a water-oil (W/O) production method for long term delivery of a GnRH antagonist.

In an exemplary embodiment, the composition achieves a therapeutic effect within 24 hours and maintains therapeutic effect for at least 90 days for >95% percent of treated patients.

In a particular embodiment, the composition is in the form of a hydrogel. In another particular embodiment, the composition is in the form of microspheres.

In one aspect, the composition of the invention is an injectable composition, which is administered with one injection or two injections administered concurrently or consecutively, with a total injection volume, for example, less than 4 ml. Injections may be subcutaneous or intramuscular.

In another aspect, the invention relates to a method for extending the release of a GnRH antagonist (e.g., cetrorelix) in a subject for a period ranging from about 1 month to about 6 months. The method comprising administering to the subject a composition comprising cetrorelix and a multi-block copolymer. The multi-block copolymer comprising poly-lactic co-glycolic acid (PLGA) in a lactide:glycolide molar ratio between 1:1, 1:0, and 0:1. Wherein cetrorelix is present in an amount of 5%-90% of the weight of the composition, and the multi-block copolymer is present in an amount of 10%-50% of the weight of the composition.

In another aspect, the invention relates to a method of maintaining a therapeutic level of a GnRH antagonist (e.g., cetrorelix) in a subject for a period ranging from about 1 month to about 6 months. The method comprising administering to the subject a composition comprising cetrorelix and a polymer. The polymer comprising poly-lactic co-glycolic acid (PLGA) in a lactide:glycolide molar ratio between 1:1, 1:0, and 0:1. Wherein cetrorelix is present in an amount of 5%-90% of the weight of the composition, and the polymer is present in an amount of 10%-50% of the weight of the composition.

In another aspect, the composition of the invention has an encapsulation efficiency greater than 70% and a consistent release of the active agent from the drug delivery vehicle with no more than 25% variation. In yet another aspect, the composition of the invention releases the active agent from the drug delivery vehicle with >85% intact in the active form over the entire duration of release.

Other features and advantages of the present invention will become apparent from the following detailed description, examples, and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows in vitro release of cetrorelix (CRX) from microspheres (MSP) composed of different polymers and carriers.

FIG. 2 shows in vitro release of cetrorelix (CRX) from microspheres (MSP) composed of different polymers loaded with cetrorelix prepared in 35% Acetic acid/65% H₂O aqueous phase.

FIG. 3 shows daily levels of cetrorelix release from microspheres composed of different polymers.

FIG. 4A shows the morphology of cetrorelix coated 10CP10C20-D23 beads having 12.7% cetrorelix prepared using 65% Acetic Acid (HAc):25% water phase.

FIG. 4B shows the morphology of cetrorelix coated 20CP15C50-D23 beads comprising 13.4% cetrorelix.

FIG. 5 shows cetrorelix plasma concentration following administration of cetrorelix PLGA gel formulations to rats.

FIG. 6A shows long term cetrorelix plasma concentration following administration to rats of two cetrorelix salt formulations as a suspension in water.

FIG. 6B shows plasma concentration following administration to rats of two cetrorelix salt formulations as a suspension in water over the first 24 hours following administration (to show initial release).

FIG. 7A shows comparative cetrorelix plasma concentrations in rats for PLGA gel formulations and for cetrorelix salt formulations.

FIG. 7B shows comparative cetrorelix plasma concentrations in rats for PLGA gel formulations and for cetrorelix salt formulations.

FIG. 8 shows comparative cetrorelix plasma concentrations in rats after dose normalization for PLGA gel formulations and for cetrorelix salt formulations.

FIG. 9A shows rat serum testosterone levels following administration of PLGA gel formulations and for cetrorelix salt formulations over a 42 day period.

FIG. 9B shows rat serum testosterone levels following administration of PLGA gel formulations and for cetrorelix salt formulations over the first 24 hours following administration.

FIG. 10 shows cumulative cetrorelix in vitro release from PLGA gel formulations.

FIG. 11 shows cumulative cetrorelix in vitro release from RG502H/RG752H (40% PLGA)-salt microsphere formulations compared to a PLGA gel formulation.

FIG. 12 shows cumulative cetrorelix in vitro release from RG752H (40% PLGA) salt microsphere formulations compared to PLGA gel formulations.

FIG. 13 shows cumulative cetrorelix in vitro release from RG502H/RG752H (40% PLGA)-salt microsphere formulations.

FIG. 14 shows cetrorelix plasma concentration following administration to rats of cetrorelix PLGA gel formulations (VH-022-001, VH-023-002 and VH-024-001) for up to 119 days (17 weeks) and of cetrorelix salt formulations (Groups 4 and 5) for ≥49 days (7 weeks). The percent day 1 release ranged from 9 to 13% of the total cetrorelix release.

FIG. 15A shows average rat serum testosterone levels following administration of various cetrorelix PLGA gel formulations (VH-022-001, VH-023-002 and VH-024-001) for up to 19 weeks and cetrorelix salt formulations (Groups 4 and 5). PLGA gel formulations showed undetectable testosterone level for ≥133 days (19 weeks). Group 4 salt formulations showed undetectable testosterone level for greater than 1 week.

FIG. 15B shows rat serum testosterone levels following administration of various cetrorelix gel formulations and salt formulations, as indicated in FIG. 15A, over the first week following administration, showing undetectable testosterone levels for all formulations after the first 24 hours.

FIGS. 16A and 16B show testosterone serum levels. FIG. 16A shows the testosterone serum level for several gel formulations in rats after 12 weeks. FIG. 16B is zoomed in for the 1^{st} day of FIG. 16A showing undetectable testosterone levels after 24 hours.

FIG. 17 shows cetrorelix levels in rats after 12 weeks for the gel formulations of FIG. 16.

FIGS. 18A and 18B show serum cetrorelix data in dogs. FIG. 18A shows serum cetrorelix data for 19.9% RG752H, 30% RG752H, and 30% R202H solutions. FIG. 18B shows cetrorelix level 1 week prior to dosing. LLOQ (ng/ml)=0.5. Animal D0103 (Group 2) vocalized and struggled during dose administration. As a result, the dose was delivered as 2 injections. The animal did receive the full dose as no formulation appeared to have been lost due to the animal struggling.

FIGS. 19A-19C show a comparison of rat and dog PK results for cetrorelix in gel formulations (up to 39 weeks). FIG. 19A shows 2^{nd} rat study results with cetrorelix levels above 1 ng/ml for up to 39 weeks. FIG. 19B shows 1^{st} dog study results with cetrorelix levels above 1 ng/ml for up to 26 weeks. FIG. 19C shows the formulation compositions and pharmacokinetic data for dogs and rats. Similar formulations show similar results in both species.

FIGS. 20A-20B show serum testosterone data. FIG. 20A shows serum testosterone data for 19.9% RG752H, 30% RG752H, and 30% R202H solutions. FIG. 20B shows testosterone level 1 week prior to dosing. LLOQ (ng/ml)=0.01. Animal D0103 (Group 2) vocalized and struggled during dose administration. As a result, the dose was delivered as 2 injections. The animal did receive the full dose as no formulation appeared to have been lost due to the animal struggling.

FIGS. 21A-21C show rat and dog study results for testosterone (up to 39 weeks). FIG. 21A shows 2^{nd} rat study results with undetectable testosterone for greater than 30 weeks. FIG. 21B shows 1^{st} dog study results with undetectable testosterone for greater than 12 weeks. FIG. 21C shows the formulation data of the cetrorelix, the dosing of the experiment, the maximum serum concentration of the dose, and the area under the curve of the dose.

FIGS. 22A and 22B show 1^{st} dog study for group 1 (19.9% RG752H solution). FIG. 22A shows serum cetrorelix level. FIG. 22B shows serum testosterone level. These figures show that testosterone level goes up once cetrorelix level drops less than 2 ng/mL.

FIGS. 23A and 23B show 1^{st} dog study for group 2 (30% RG752H solution). FIG. 23A shows serum cetrorelix level. FIG. 23B shows serum testosterone level. These figures also show that testosterone level goes up once cetrorelix level drops less than 2 ng/mL.

FIGS. 24A and 24B show 1^{st} dog study for group 2 (30% R202H solution). FIG. 24A shows serum cetrorelix level. FIG. 24B shows serum testosterone level. These figures also show that testosterone level goes up once cetrorelix level drops less than 2 ng/mL.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

The invention relates to a controlled release composition comprising a gonadotropin-releasing hormone (GnRH) antagonist in combination with one or more polymers and/or salts. The GnRH antagonist and polymer composition may be stored in solution or suspension in a solvent suitable for both to create a stable formulation for storage in a prefilled syringe.

The composition may include any suitable a GnRH antagonist, known to one of skilled in the art. GnRH is also known as follicle-stimulating hormone-releasing hormone (FSH-RH), luteinizing hormone-releasing hormone (LHRH), gonadoliberin, and by various other names, known to one of skilled in the art.

In a particular embodiment, the GnRH antagonist is cetrorelix, abarelix, degarelix, ganirelix, ozarelix, taverelix, antarelix, or iturelix.

In one aspect, provided herein is a composition. The composition comprising a therapeutically effective amount of a GnRH antagonist in combination with a multi-block copolymer. Wherein the multi-block copolymer is polyglycolide (PLG), polylactide (PLA), or poly-(lactic co-glycolic acid) (PLGA). Wherein the composition releases the GnRH antagonist for a long term. In a particular embodiment, the composition releases the GnRH antagonist for at least 180 days. In certain embodiments, the composition release the GnRH antagonist (e.g., cetrorelix) for at least 120 days. In certain embodiments, the composition achieves a therapeutic effect within 24 hours and maintains the therapeutic effect for at least 120 days.

In particular embodiments, the invention relates to a composition for a long-term release of cetrorelix. The composition comprising a biodegradable multi-block copolymer, a solvent, and a therapeutically effective amount of cetrorelix, wherein the long-term release is a duration of at least six months. The composition may have a release term of about three months, six months, nine months, or about 12 months. Preferably, the composition has a release term of about nine months or more.

In another aspect, provided herein is a composition. The composition comprising: a therapeutically effective amount of a GnRH antagonist in combination with a non-PLGA multi-block copolymer, wherein the composition releases the GnRH antagonist for a long term. Non-PLGA multi-block copolymer are well known in the art. Examples of a non-PLGA multi-block copolymer include, for example, but are not limited to, polyethyleneglycol (PEG), PLG, PLA, polybutylene terephthalate (PBT), poly(epsilon-caprolactone) (PCL), dioxanone, butanediisocyanate, butanediol polyoxyethylene, polypropylene, polyoxypropylene, polystyrene, poly methyl methacrylate, or a block copolymer which additionally incorporates one more novel amphiphilic, hydrophilic, or hydrophobic component. In another aspect, a non-PLGA block polymer comprises a blend of two or more multi-block copolymer types capable of releasing therapeutically effective amount of GnRH antagonists.

In one aspect, the composition is a flowable composition capable of forming an in situ implant in a subject. In one example, the composition includes a biodegradable thermoplastic multi-block copolymer, a biocompatible solvent; and a GnRH antagonist.

In another aspect, the invention relates to a flowable composition, the composition comprising: (a) a biodegradable thermoplastic multi-block copolymer that is at least substantially insoluble/stable in aqueous medium or body fluid; (b) a biocompatible polar aprotic solvent, wherein the biocompatible polar aprotic solvent is miscible to dispersible in aqueous medium or body fluid; and (c) a therapeutically effective amount of cetrorelix.

The biodegradable thermoplastic multi-block copolymer can be substantially insoluble/stable in aqueous medium or body fluid. Biodegradable thermoplastic multi-block copolymer are well known in the art and fully described in U.S. Pat. Nos. 6,565,874; 5,324,519; 4,938,763; 5,702,716; 5,744,153; and 5,990,194, which are incorporated by reference herein in their entirety. In one embodiment, biodegradable thermoplastic multi-block copolymer is a polyester, for example, including but not limited to, a polylactide, a polyglycolide, a polycaprolactone, a copolymer thereof, a terpolymer thereof, or any combination thereof.

The type, amount, and molecular weight, of biodegradable thermoplastic multi-block copolymer present in the composition may depend upon one or more desired properties of the controlled release implant.

Examples of types of biodegradable thermoplastic polyesters are well known in the art and fully described in U.S. Pat. No. 6,565,874. In a particular embodiment, the suitable biodegradable thermoplastic polyester is 1:1 poly (DL-lactide-co-glycolide) having a carboxy terminal group or is 3:1 poly (DL-lactide-co-glycolide) with a carboxy terminal group that is protected. Other suitable copolymers, known to one of skilled in the art, can also be used.

In another aspect, the PLGA multi-block copolymers in the compositions of the present invention may have lactide:glycolide weight ratio ranging from about 1:1 to about 1:0. In particular embodiments, the lactide to glycolide ratio is about, 1:1, 11:9, 3:2, 13:7, 7:3, 3:1, 4:1, 5.67:1, 9:1, or 20:1.

The PLGA polymers the compositions of the present invention may comprise a mixture of two or more PLGA multi-block copolymers each having a different glycolide and lactide fractions. For example, the mixture may include a first PLGA polymer having equal amount of glycolide and lactide (RG502H) and a second PLGA polymer having 25% glycolide and 75% lactide (RG752H). The proportions of the first PLGA polymer and the second PLGA polymer may vary, for example the ratio of RG502H to RG752H can range from about 1:0 to about 0:1.

The amount of biodegradable thermoplastic multi-block copolymer, in the composition, can be any suitable amount, known to one of skilled in the art. The amount, in the composition, may range from about 10 wt. % to about 80 wt. %; from about 20 wt. % to about 60 wt. %; from about 25 wt. % to about 55 wt. %; from about 30 wt. % to about 50 wt. %; or from about 35 wt. % to about 45 wt. %. In a particular embodiment, the amount is approximately 10, 20, 25, 30, 35, 40, 45, 50, 55, 60, 65, 70, or 80 wt. %.

The molecular weight of biodegradable thermoplastic polymer, in the composition, can be any suitable molecular weight, known to one of skilled in the art. The molecular weight may range from about 10,000 to about 50,000; from about 15,000 to about 45,000; from about 20,000 to about 40,000; or from about 20,000 to about 30,000. In a particular embodiment, the molecular weight is approximately 10,000, 15,000, 20,000, 25,000, 30,000, 35,000, 40,000, 45,000, or 50,000.

In particular embodiments, the biodegradable thermoplastic polyester has an average molecular weight ranging from about 15,000 to about 45,000 or from about 20,000 to about 35,000.

The biocompatible solvent can be a biocompatible solvent. The biocompatible solvent can be a biocompatible polar aprotic solvent. In one embodiment, the solvent is miscible to dispersible in aqueous medium or body fluid. Suitable polar aprotic solvents are well known in the art and fully described in, for example, in Aldrich Handbook of Fine Chemicals and Laboratory Equipment, Milwaukee, Wis. (2000) and U.S. Pat. Nos. 6,565,874, 5,324,519; 4,938,763; 5,702,716; 5,744,153; and 5,990,194.

In one aspect, the solvent of the invention diffuses into body fluid so that the flowable composition coagulates or solidifies. In another aspect, the solvent of the invention is biodegradable. In another aspect, the solvent of the invention is biocompatible. In yet another aspect, the solvent of the invention is non-toxic. As set forth in U.S. Pat. No. 6,565,874, examples of suitable polar aprotic solvents include polar aprotic solvents having an amide group, an ester group, a carbonate group, a ketone, an ether, a sulfonyl group, or a combination thereof.

In one embodiment, the polar aprotic solvent is N-methyl-2-pyrrolidone, 2-pyrrolidone, N,N-dimethylformamide, N,N-dimethylacetamide, dimethyl sulfoxide, propylene carbonate, caprolactam, triacetin, benzyl benzoate, propylene glycol, or any combination thereof. In another embodiment, the polar aprotic solvent is N-methyl-2-pyrrolidone.

As set forth in U.S. Pat. No. 6,565,874, the polar aprotic solvent can be present in any suitable amount. The type and amount of biocompatible polar aprotic solvent present in the composition may depend upon the desired properties of the controlled release implant. In a particular embodiment, the type and amount of biocompatible polar aprotic solvent influences the initial release rate and the length of time in which the GnRH antagonist is released from the controlled release implant.

In various embodiments of the long-term release GnRH antagonist composition (e.g., cetrorelix), the solvent may be present at the concentration ranging from about 10% to about 30% (w/w). For example, the cetrorelix may be present at a concentration ranging from about 5% to about 90% (w/w). Alternatively, cetrorelix may be present in an amount of about 50 mg to about 300 mg or 50 mg to about 150 mg per dosage.

In certain embodiments, the composition may further comprise a salt, i.e., a salt of the GnRH antagonist (e.g., cetrorelix). Examples of a salt, include but are not limited to calcium pamoate (Ca pamoate), sodium pamoate (Na pamoate) and calcium citrate (Ca citrate). In the gel compositions, the cetrorelix may be present as a salt, i.e., cetrorelix salt. Examples of salts include, but are not limited to, sulfate, citrate, acetate, oxalate, chloride, bromide, iodide, nitrate, bisulfate, phosphate, acid phosphate, carbonate, bicarbonate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, and pamoate. Preferred examples of cetrorelix salts include cetrorelix carbonate, cetrorelix acetate, cetrorelix benzoate, cetrorelix formate, and cetrorelix citrate. Most preferred cetrorelix salt is cetrorelix acetate.

In another aspect, the invention is a method of preparing a flowable composition, the method comprising: mixing a biodegradable thermoplastic multi-block copolymer, a biocompatible solvent; and a GnRH antagonist. The mixing is performed for a sufficient period of time effective to form the flowable composition for use as a controlled release implant.

In yet another aspect, the invention is an implant formed in situ by the process of injecting the composition of the invention to a subject; allowing the solvent, in the composition, to dissipate to produce a solid biodegradable implant.

In yet another aspect, the invention is a method of forming an implant in situ in a subject, the method comprising the steps of: injecting the composition of the invention to a subject; and allowing the solvent, in the composition, to dissipate to produce a solid biodegradable implant.

In one example, the flowable composition of the invention comprises a flowable delivery system such as an Atrigel® system comprising a multi-block copolymer, a water-soluble organic solvent, and a bioactive agent, for example, a GnRH antagonist.

ATRIGEL® is a registered Trademark of Tolmar Pharmaceuticals, Fort Collins, Colo. found in H. B. Ravivarapu, K. L. Moyer, R. L. Dunn, International Journal of Pharmaceutics, 194 (2000) 181-191, which is incorporated in its entirety. ATRIGEL®, unless otherwise specified, comprises PLGA dissolved in 1-methyl-2-pyrrolidinone (NMP).

The invention relates to a controlled release composition comprising a gonadotropin-releasing hormone (GnRH) antagonist (e.g., cetrorelix) loaded in a multi-block copolymer.

In one aspect, the inventor relates to a multi-block copolymer composition having a gonadotropin-releasing hormone (GnRH) antagonist (e.g., cetrorelix) as a bioactive agent. The multi-block copolymer compositions are well known and fully described in U.S. Pat. Nos. 8,481,651; 8,674,032; 8,674,033; and 9,364,442 and U.S. Application Publications 2013/0209568; 2013/0273284; and 2014/0199385, and PCT International Application Publications WO2005068533; WO2004007588; WO2012005594; and WO2013015685, all of which are incorporated by reference herein in their entirety.

The multi-block copolymer comprises one or more hydrolysable segments. In one embodiment, the multi-block copolymer comprises one or more randomly arranged hydrolysable segments. In another embodiment, the multi-block copolymer comprises one or more non-randomly arranged hydrolysable segments. In yet another embodiment, the multi-block copolymer comprises one or more alternatingly arranged hydrolysable segments. In yet another embodiment, the multi-block copolymer comprises one or more non-alternatingly arranged hydrolysable segments.

In some embodiments, the segments can be randomly and non-alternatingly connected to each other by multi-functional chain extenders.

In one example, the multi-block copolymer is amorphous at human body conditions.

In an exemplary embodiment, the multi-block copolymer has a glass transition temperature below body temperature at human body conditions.

In another aspect, the multi-block copolymer includes pre-polymer A, pre-polymer B, or a combination thereof. In one embodiment, pre-polymers A and B are composed of different monomers. In another embodiment, pre-polymers A and B are composed of the same monomers but in a different amount. In yet another embodiment, the pre-polymers are composed of the same monomers but with a different initiator in order to obtain the multi-block copolymers of the present invention. In one embodiment the multi-block copolymer is comprised of pre-polymers A and B which are the same monomer with the same initiators.

Pre-polymers A and B can be selected in such a way that the segments would exhibit significantly different properties, for example, but not limited to thermal, degradation and hydrophilic properties.

The pre-polymers A or B may comprise a hydrolysable polyester, poly ether ester, polycarbonate, polyester carbonate, polyanhydride or copolymers thereof. The pre-polymers can be derived from cyclic monomers such as lactide (L, D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione).

In one embodiment, the pre-polymer includes an ester linkage. In another embodiment, pre-polymer includes a carbonate linkage. In yet another embodiment, pre-polymer includes an anhydride linkage. In some embodiments, pre-polymer optionally comprises a polyether group. In an exemplary embodiment, polyether is present as an additional pre-polymer.

In one example, pre-polymer comprises a reaction product of an ester forming monomer. The ester forming monomer can be selected from the group consisting of diols, dicarboxylic acids and hydroxycarboxylic acids.

In another example, pre-polymer comprises reaction products of at least one suitable cyclic monomer with at least one non-cyclic initiator. The non-cyclic initiator can be selected from the group consisting of diols, dicarboxylic acids and hydroxycarboxylic acids.

Examples of cyclic monomers include, but are not limited to, glycolide, lactide (L, D or DL), ε-caprolactone, δ-valerolactone, trimethylene carbonate, tetramethylene carbonate, 1,4-dioxane-2-one (para-dioxanone), 1,5-dioxepane-2-one and cyclic anhydrides.

In some embodiments, the pre-polymer comprises at least two different cyclic monomers. In one example, pre-polymer comprises glycolide and ε-caprolactone in a 1:1 weight ratio. In another example, pre-polymer comprises glycolide and lactide in a 1:1 weight ratio.

Examples of non-cyclic initiator include, but are not limited to, succinic acid, glutaric acid, adipic acid, sebacic acid, lactic acid, glycolic acid, hydroxybutyric acid, ethylene glycol, diethylene glycol, 1,4-butanediol and 1,6-hexanediol.

Examples of polyether groups include, but are not limited to, PEG (polyethylene glycol), PEG-PPG (polypropylene glycol), PTMG (polytetramethylene ether glycol) and combinations thereof. In a particular embodiment, the polyether group is PEG. PEG can be an initiator for ring-opening polymerization. PEG with any suitable molecular weight can be used, for example, a molecular weight between 150-40000. In one embodiment, each of pre-polymers A and B has a number average molecular weight between 300 and 30000.

In a particular embodiment, the composition comprises a polyethylene glycol (PEG). Any suitable PEG known to one of skilled in the art can be used. In an exemplary embodiment, PEG is polyethylene glycol 200, polyethylene glycol 300, or methoxy polyethylene glycol 350.

The chain-extender of the invention can be any suitable multifunctional chain extender, known to one of skilled in the art. In one embodiment, the pre-polymers are linked by the di-functional chain-extender. Examples of di-functional chain-extender include, for example, but are not limited to, a diisocyanate chain-extender, a diacid and a diol compound.

The amount of pre-polymer, in the composition, can be any suitable amount, known to one of skilled in the art. The amount, in the composition, may be of about 10-90 wt. %.

The methods for synthesis of pre-polymers and multi-block copolymer compositions are well known and fully described in U.S. Pat. Nos. 8,481,651; 8,674,032; 8,674,033; and 9,364,442 and U.S. Application Publications 2013/0209568; 2013/0273284; and 2014/0199385, and PCT International Application Publications WO2005068533; WO2004007588; WO2012005594; and WO2013015685.

The intrinsic viscosity also may vary depending on one or more desired properties. In some embodiment, the intrinsic viscosity is larger than about 0.1 dl/g and less than about 6 dl/g. In one embodiment, the intrinsic viscosity lies between about 0.2-4 dl/g, more preferably between 0.4-2 dl/g.

In another aspect, the invention relates to phase separated multi block copolymers. The term “phase-separated,” as used herein, may refer to a system, for example, a copolymer having two or more different pre-polymers, of which at least two are incompatible with each other at temperatures of 40° C. or below (when kept at body conditions). As a result, the pre-polymers do not form a homogeneous mixture when combined as a physical mixture or chemical mixture.

The phase separated multi block copolymers are well known in the art and fully described in U.S. Pat. Nos. 9,364,442 and 8,674,033, and PCT International Application Publications WO2012005594 and, WO2004007588. The phase-separated quality of the copolymers of the present invention is reflected in the profile of the glass transition temperature (Tg), melting temperature (Tm), or a combination thereof. For example, the phase-separated copolymers are characterized by at least two-phase transitions, each of which is related to (but not necessarily identical to) the corresponding Tg or Tm values of the prepolymers which are comprised in the copolymer. In an exemplary embodiment, the multi-block copolymer is a phase separated multiblock copolymer, comprising: one or more segments of a pre-polymer A (e.g., a linear soft biodegradable pre-polymer A) having a glass transition temperature (T_{g}) lower than 37° C.; and one or more segments of a pre-polymer B (e.g., a linear hard biodegradable pre-polymer B) having a melting point temperature (Tₘ) ranging from about 40° C. to about 100° C.

In another aspect, the invention relates to a composition, the composition comprising: a therapeutically effective amount of a cetrorelix in combination with a multi-block copolymer, wherein the multi-block copolymer comprises randomly or non-alternatingly arranged hydrolysable segments, wherein each segment comprises pre-polymer A or pre-polymer B, and wherein the segments are operably linked to each other by a multifunctional chain extender. In an exemplary embodiment, the multi-block copolymer is a phase separated multiblock copolymer, comprising: one or more segments of a linear soft biodegradable pre-polymer A having a glass transition temperature (T_{g}) lower than 37° C.; and one or more segments of a linear hard biodegradable pre-polymer B having a melting point temperature (Tₘ) of 40-100° C.

The multi-block copolymer compositions may be in any suitable form, for example, in the form of implant, microspheres, microrods, microparticles, injectable gel formulation, coatings or membranes or devices, or any other form known in the art.

In a particular embodiment, the composition forms of a hydrogel. In another particular embodiment, the composition is in the form of microspheres. In particular embodiments, the microspheres are loaded with a GnRH antagonist (e.g., cetrorelix) and without salt.

In another aspect, the invention relates to the use of salt bridges or cyclization of the active agent either as a primary drug delivery technique or in combination with another drug delivery vehicle using compounds that include, but are not limited to, lanthionine, dicarba, hydrazine, or lactam bridges. The formation of salt bridges for linking through non-covalent bonds are well known in the art and fully described in PCT International application publications WO2009/155257 and WO 2012/163519, which are incorporated by reference herein in their entirety.

In another aspect, the invention relates to the use of micronization or stabilizing adjuvants for a long-term delivery of a GnRH antagonist. Micronization techniques are well known in the art and fully described, for example, in PCT International Application Publication WO2011/034514 and U.S. Application Publication US2014/0219954, all of which are incorporated by reference herein in their entirety. Stabilizing adjuvants are also well known in the art and fully described, for example, in U.S. Pat. No. 7,611,709, which is incorporated by reference herein in its entirety.

In another aspect, the invention relates to the use of a solid-in-oil-in-water (S/O/W), a water-in-oil-in water (W/O/W), or a water-oil (W/O) production method for long term delivery of a GnRH antagonist. These methods are well known in the art and fully described, for example, in PCT International Application Publications WO2015/145353; WO2003/099262; and WO2007/129926 and U.S. Application Publications US2002/0055461; US2008/0268004; and US2010/0019403, which are incorporated by reference herein in their entirety.

In an exemplary embodiment, the composition achieves a therapeutic effect within 24 hours and maintains therapeutic effect for at least 180 days in, for example, >95% percent of treated patients. In another embodiment, the composition achieves a therapeutic effect within 24 hours and maintains therapeutic effect for at least 133 days in, for example, >95% percent of treated patients. In alternate embodiments, the composition achieves a therapeutic effect within 7 days and maintains the therapeutic effect for at least 90 days in, for example, >95% percent of treated patients.

Microspheres for sustained release of therapeutically active agents and methods of their preparations are well known in the art (see e.g. U.S. Pat. Nos. 6,458,387 and 9,381,159 which are incorporated by reference herein in their entirety). The microspheres typically comprise a matrix formed of biodegradable polymer. In some embodiments, the inner matrix diffuses through the outer surface under appropriate conditions. In some embodiments, the outer surface not only enables aqueous fluids to enter the microsphere, but also enables solubilized drug and polymer to exit the microsphere. The microspheres are made to release drug and polymer from the interior of the microsphere when placed in an appropriate aqueous medium, such as body fluids or a physiologically acceptable buffer under physiological conditions over a prolonged period of time, thereby providing sustained release of a drug. In one embodiment, the microspheres are made to release a drug without an initial burst or rapid drug release.

The microspheres have a generally uniform size (substantially spherical) and shape, with each preparation having a narrow size distribution. Microspheres range in size from about 0.5 microns to about 100 microns, depending upon the fabrication conditions. The characteristics of the microspheres may be altered during preparation by manipulating the water-soluble polymer concentration, reaction temperature, pH, concentration of therapeutic agent, crosslinking agent, and/or the length of time the macromolecule is exposed to the crosslinking agent and/or the energy source. In one example, microspheres are suitable for oral or parenteral administration; mucosal administration; ophthalmic administration; intravenous, subcutaneous, intra articular, or intramuscular injection; administration by inhalation; or topical administration.

In one embodiment the microsphere size is between 20-90 μm. In another embodiment the microsphere size is between 30-80 μm. In another embodiment the microsphere size is between 45-70 μm.

The amount of polymer matrix, in the microsphere composition, can be any suitable amount, known to one of skilled in the art. The amount in the microsphere composition, may range from about 10 wt. % to about 50 wt. %; from about 20 wt. % to about 40 wt. %; from about 25 wt. % to about 35 wt. %. In a particular embodiment, the amount is approximately 10, 20, 25, 30, 35, 40, 45, or 50 wt. %.

The amount of therapeutic molecule in the microsphere ranges from about 1 wt. % to about 90 wt. %, from about 1 wt. % to about 40 wt. %, from about 3 wt. % to about 30 wt. %, from about 5 wt. %. to about 20 wt. %, from about 10 wt. %. to about 15 wt. %. In a particular embodiment, the amount is approximately 1, 3, 5, 7, 9 10, 15 20, 25, 30, 35, 40, 45, 50, 60, 70, 80, or 90 wt. %.

In one embodiment the therapeutic molecule is loaded between 5% and 20% weight/weight. In another embodiment the therapeutic molecule is loaded between 7% and 13% weight/weight.

In one embodiment the Cₘₐₓ (maximum concentration in the blood stream) is between 100 ng/ml and 400 ng/ml, or between 200 ng/ml and 350 ng/ml. More preferably between 275 ng/ml and 325 ng/ml.

In one embodiment the area under the curve (the curve of the drug concentration in blood plasma vs. time) (AUC) is between 10000 ng/mL*hr and 30000 ng/mL*hr. In another embodiment the AUC is between 12500 ng/mL*hr and 25000 ng/mL*hr. In another embodiment the AUC is between 15000 ng/mL*hr and 20000 ng/mL*hr.

In one embodiment the amount of cetrorelix released after one-and-a-half months is between 5% and 25% of the total amount of cetrorelix. In another embodiment the amount of cetrorelix released at this time is between 10% and 20% of the total amount of cetrorelix. In another embodiment the amount of cetrorelix released is between 12% and 15% of the total amount of cetrorelix.

In one embodiment the polymer is loaded at 15%-45% weight/weight. In another embodiment the polymer is loaded at 25%-35% weight/weight.

In one aspect, the composition of the invention is an injectable composition, which is administered with one injection or two injections administered either concurrently or consecutively, with a total injection volume of less than 4 mL. The injection may be subcutaneous or intramuscular. The GnRH antagonist and polymer may be stored in solution or suspension in a solvent suitable to create a stable formulation for storage in a prefilled syringe. The syringe may contain a GnRH antagonist, such as cetrorelix, in an amount of about 50 mg to about 300 mg or about 50 mg to about 150 mg.

The invention also relates to a kit, wherein the kit comprising: the composition of the invention.

In another aspect, the composition of the invention is prepared with over 75% encapsulation/incorporation efficiency, and exhibits consistent release of the active agent from the drug delivery vehicle with no more than 25% variation. In yet another aspect, the composition of the invention releases the active agent from the drug delivery vehicle with >85% intact over the entire duration of release.

In a further aspect, the invention relates to a method of extending release of a pharmaceutical agent (e.g. cetrorelix) in a subject for a period ranging from about 1 month to about 6 months or about 1 month to about 9 months or greater than 9 months. The method comprising administering to the subject a composition of the invention (e.g. microspheres or gel). In another aspect, the invention relates to a method of extending the release of a pharmaceutical agent (e.g. cetrorelix) in a subject for a period of at least 90 days, the method comprising administering to the subject a composition of the invention (e.g. microspheres).

In a yet further aspect, the invention relates to a method of maintaining an effective level of a therapeutic agent (e.g. cetrorelix) in a subject for a period ranging from about 1 month to about 6 months or from about 1 month to about 9 months or greater than 9 months. The method comprising administering to the subject a composition of the invention (e.g. microspheres or gel). In another aspect, the invention relates to a method of maintaining an effective level of a therapeutic agent (e.g. cetrorelix) in a subject for a period at least 90 days, the method comprising administering to the subject a composition of the invention (e.g. microspheres).

In yet another aspect, the invention relates to a method for treating a disease or condition associated with GnRH, the method comprising administering a therapeutically effective amount of the composition of the invention, thereby treating the disease in the subject.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human, however non-human mammals, including transgenic mammals, can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

The compositions of the invention described herein can be used to treat any GnRH associated disease or condition that could be treated by GnRH antagonist in humans or animals. Examples of treatments, for diseases or conditions treated by the compositions of the invention include, for example, but are not limited to, suppression of testosterone production, as well as suppression of the hormones FSH and/or LH for the treatment of prostate cancer, especially hormone sensitive prostate cancer, and benign prostatic hyperplasia, directly blocking GnRH receptors on prostate cells for treatment of prostate cancer and benign prostatic hyperplasia, controlled ovarian stimulation for assisted reproductive techniques, treatment of uterine myoma, suppression of ovarian function while undergoing chemotherapy, treatment of breast cancer, treatment of ovarian cancer, male contraception, and female contraception.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented. The methods of treatment described herein can be used to treat any suitable mammal, including primates, such as monkeys and humans, horses, cows, cats, dogs, rabbits, elk, deer and rodents such as rats and mice. In one embodiment, the mammal to be treated is human.

“Administration” to a subject is not limited to any particular delivery system and may include, without limitation, parenteral (including subcutaneous, intravenous, intramedullary, intraarticular, intramuscular, or intraperitoneal injection).

The composition of the invention may be administered parenterally (e.g., intravenous, subcutaneous, intraperitoneal, and intramuscular). Further, the composition of the invention may be administered by intravenous infusion or injection. The composition of the invention may be administered by intramuscular or subcutaneous injection. In some embodiments, the composition of the invention may be administered surgically. As used herein, a “composition” refers to any composition that contains a pharmaceutically effective amount of one or more active ingredients (e.g., a GnRH antagonist). The composition, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

All patents and literature references cited in the present specification are hereby incorporated by reference in their entirety.

### EXAMPLES

### Example 1

Poly(DL-lactide-co-glycolide) with 1:1 ratio of lactide to glycolide is dissolved in a suitable solvent to prepare an Atrigel® polymer solution. This solution is then filled into a syringe with a female luer lock fitting.

Each GnRH antagonist (ozarelix, degarelix, cetrorelix, or ganirelex) is then dissolved in water or other solvents and filled into a syringe with a male luer-lock fitting.

Prior to administration, the two syringes are coupled, and the contents are mixed back and forth between the two syringes for multiple cycles. After thorough mixing, the formulation is drawn back into the syringe with the male coupling.

Then, the two syringes are separated and a needle is attached. The contents of the syringe is then subcutaneously injected into subjects. A total injection volume would be less than 4 mL per syringe and per injection.

Serum is then collected and analyzed. The GnRH antagonist composition may achieve a therapeutic effect within 24 hrs and maintain therapeutic effect for at least 90 days in >95% percent of treated patients.

The composition is prepared with an encapsulation efficiency of over 70% and may allow for consistent release of the active agent from the drug delivery vehicle with no more than 25% variation. The composition may release the active agent from the drug delivery vehicle with >85% intact over the entire duration of release.

### Example 2

A multi-block copolymer is provided. Each GnRH antagonist (ozarelix, degarelix, cetrorelix, or ganirelex) is loaded into the multi-block copolymer. The formulation may be in the form of microspheres.

The formulation would be subcutaneously injected into subjects. A total injection volume would be less than 4 mL.

Serum is then collected and analyzed. The GnRH antagonist composition may achieve a therapeutic effect within 24 hours and maintain therapeutic effect for at least 90 days in >95% percent of treated patients.

The composition may allow for consistent release of the active agent from the drug delivery vehicle with no more than 25% variation plus an encapsulation efficiency of over 70%. The composition may release the active agent from the drug delivery vehicle with >85% intact over the entire duration of release.

### Example 3

### Development of Cetrorelix Microspheres Formulations and Testing

Several formulations of microspheres (MSP) using different polymers and internal water phase compositions were prepared for testing cetrorelix in vitro release (IVR). The tested formulations are summarized in Table 1.

TABLE 1
Initial cetrorelix formulations

| | | | | | | CRX | |
|---|---|---|---|---|---|---|---|
| | | | | | Theoretical | Loading | |
| | | | | Microsphere | CRX | measured | |
| MSP | | | Microsphere | size (D50) | loading | by EAS | |
| Batch | Process | Polymer | morphology | (μm) | (Wt. %) | (Wt. %) | EE (%) |
| AD17-008 | W1/O/W2 | 10CP10C20-323 | Spherical, | 40 | 12.5 | 11 | 88.5 |
| | (W1 = Acetic | | monodispersed | | | | |
| | acid/H₂O | | | | | | |
| | 50/50) | | | | | | |
| RP17-004 | W1/O/W2 | 10CP10C20-323 | Spherical, | 73 | 14.3 | 13.8 | 96.5 |
| | (W1 = Acetic | | monodispersed | | | | |
| | acid/H₂O | | | | | | |
| | 35/65 pre-mix) | | | | | | |
| RP17-006 | W1/O/W2 | 10LP10L20-LL40 | Spherical, | 71 | 14.0% | 14.8 | 105.4 |
| | (W1 = Acetic | | monodispersed | | | | |
| | acid/H₂O | | | | | | |
| | 35/65 pre-mix) | | | | | | |

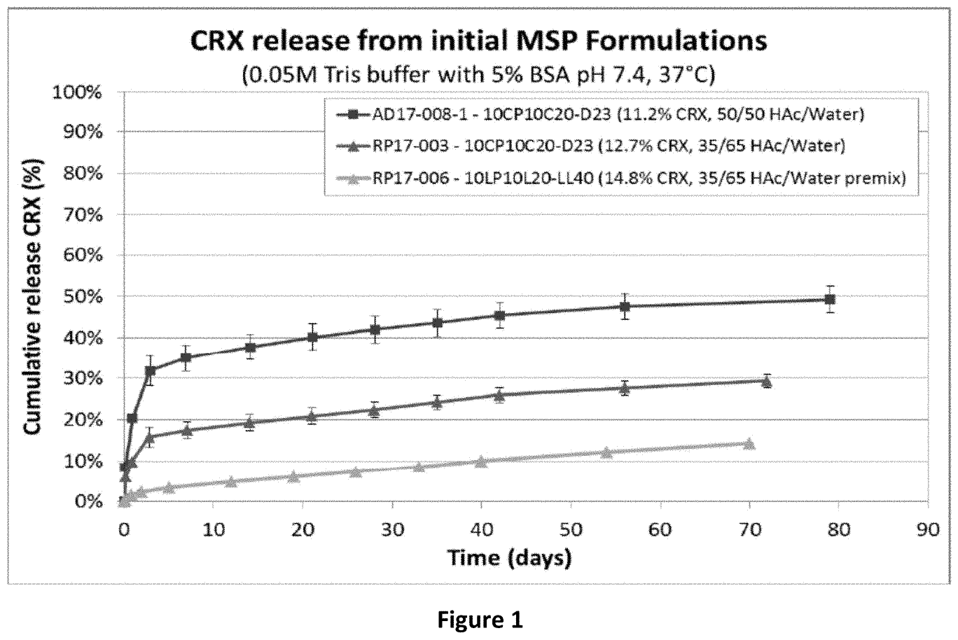

The in vitro release of cetrorelix was tested by incubating microsphere formulations listed in Table 1 in 0.05 M Tris Buffer with 5% BSA, pH 7.4 at 37° C. The results show the release was slowest when premixed 35% Acetic acid/65% H₂O as internal water phase was used (FIG. 1) in the microsphere preparation process.

To further test cetrorelix IVR, several formulations of cetrorelix-loaded microspheres using different polymers and 35% Acetic acid/65% H₂O as internal water phase were made as illustrated in Table 2.

TABLE 2
Cetrorelix formulations manufactured with optimizing primary
emulsification process (W1 = 35/65 Acetic acid/Water mixture)

| | | | | | | CRX | |
|---|---|---|---|---|---|---|---|
| | | | | | Theoretical | Loading | |
| MSP | | | Micro sphere | MSP size | CRX loading | measured by | |
| Batch | Process | Polymer | morphology | (D50) (μm) | (Wt. %) | EAS (Wt. %) | EE (%) |
| RP17-012 | W1/O/W2 | 10CP10C20-D23 | Spherical, | 82 | 15.70% | 13.5 | 85.70% |
| | | | monodispersed | | | | |
| RP17-013 | W1/O/W2 | 20CP15C50-D23 | Spherical, | 85 | 14.00% | 13.4 | 95.70% |
| | | | monodispersed | | | | |
| RP17-014 | W1/O/W2 | 20LP10C20-LL40 | Spherical, | 70 | 13.90% | 13.6 | 97.80% |
| | | | monodispersed | | | | |
| RP17-015 | W1/O/W2 | 20CP10C20-LL40 | Spherical, | 56 | 13.20% | 11.9 | 90.90% |
| | | | monodispersed | | | | |
| RP17-018 | W1/O/W2 | 30CP15C50-D23 | Spherical, | 51 | 13.80% | 9.5 | 68.40% |
| | | | monodispersed | | | | |

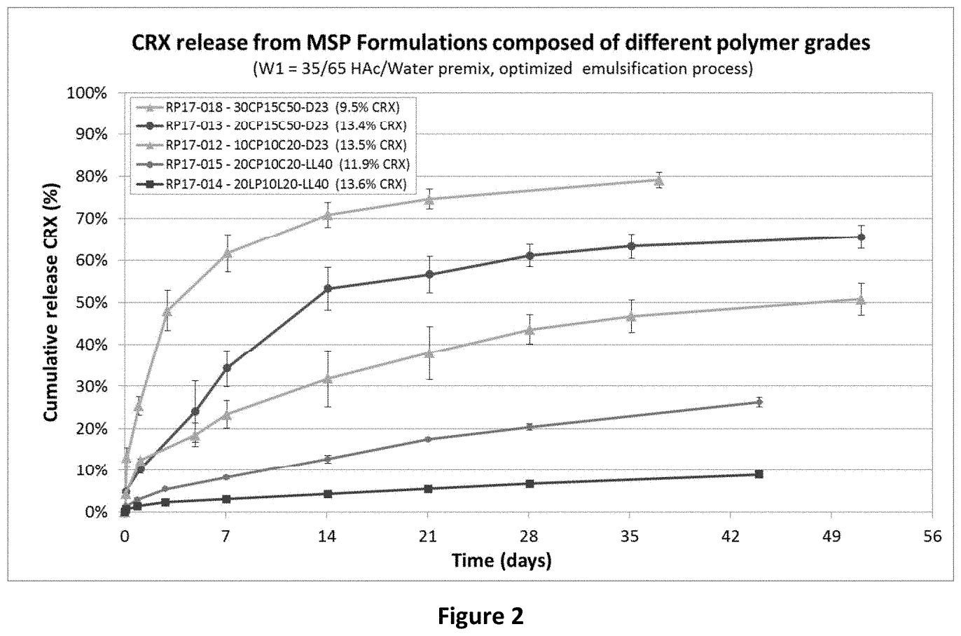

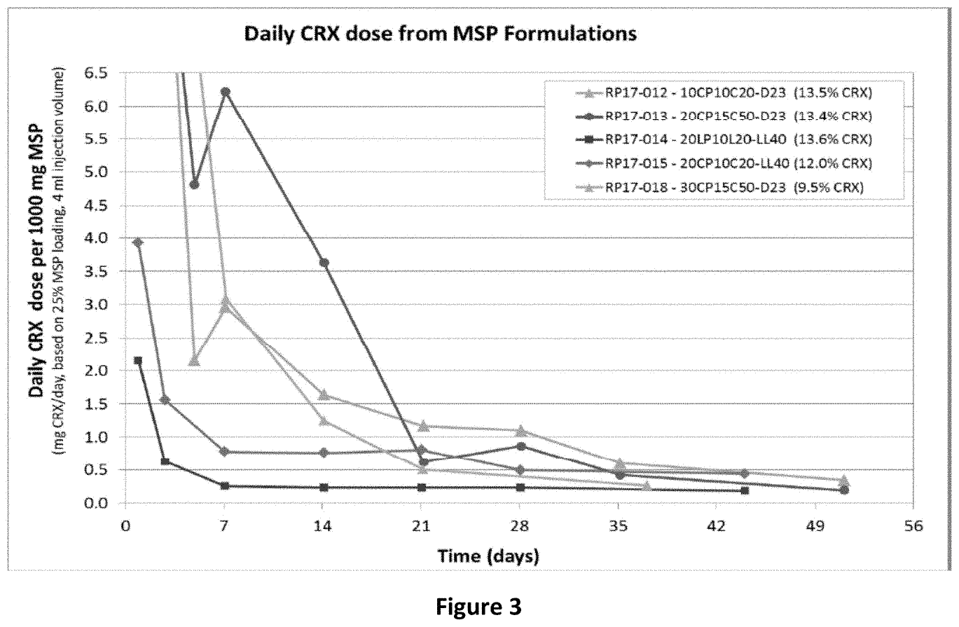

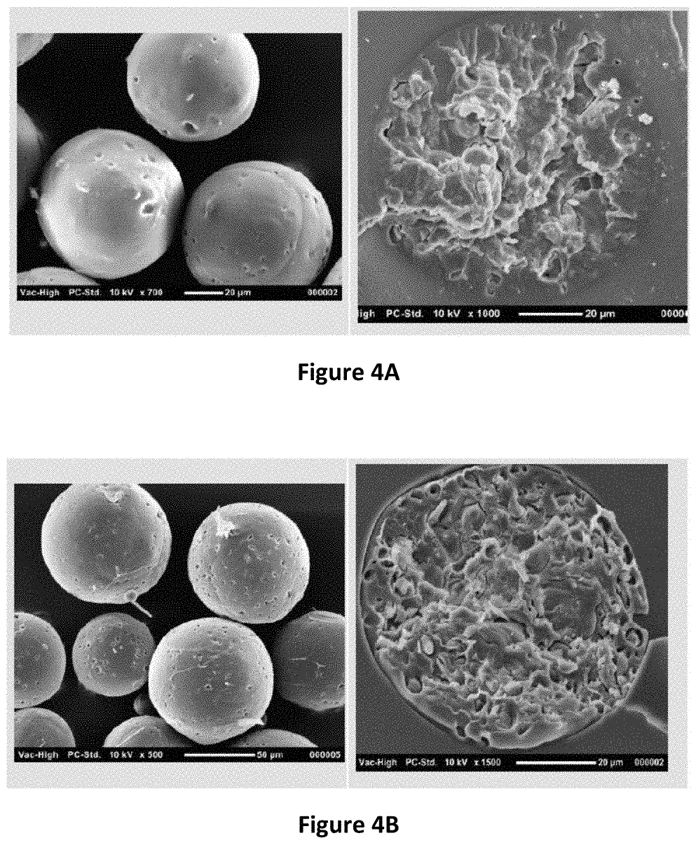

The in vitro release of cetrorelix was tested by incubating the microsphere formulations listed in Table 2 in 0.05 M Tris Buffer with 5% BSA, pH 7.4 at 37° C. The results show that the RP17-014 formulation using 20LP10C20-LL40 polymer had the slowest cetrorelix release rate, and, in addition showed linear release kinetics (FIG. 2), while RP17-012 (10CP10C20-D23) and RP17-013 (20CP15C50-D23) microsphere formulations (depicted in electron micrographs in FIG. 4A and FIG. 4B respectively) provided the highest sustained daily dose of cetrorelix (FIG. 3).

These results show that slow degrading L-Lactide based polymers with low swellability are suitable for use in cetrorelix-loaded microspheres and display linear cetrorelix release. In addition, the microsphere manufacturing process achieves cetrorelix drug load of up to 15% by weight.

### Example 4

### Cetrorelix-Loaded Gel Pharmacokinetics in Rats

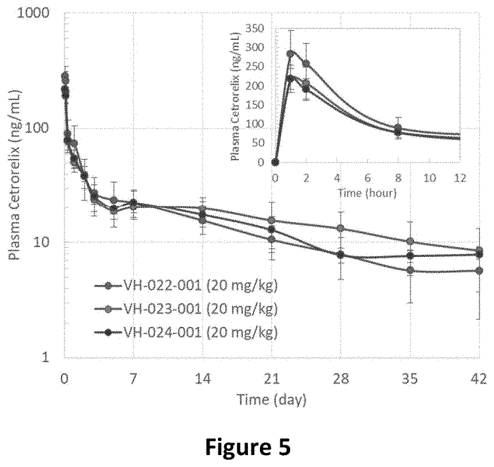

Several salt-free cetrorelix PLGA formulations having different polymer contents were tested in a rat PK Study (compositions outlined in Table 3). The preparations (<1 ml) were subcutaneously implanted into rats at a single 20 mg/kg dose and cetrorelix levels in plasma were monitored over 6 weeks. The results are summarized in Table 4. All formulations showed detectable plasma cetrorelix for greater than 42 days. Cₘₐₓ/C_{ave,42day} varied from 12.1 to 17.6 and the percentage of day 1 release related to the full 42 days ranged from 12 to 17% (FIG. 5). The amount of the polymer in the formulation (20% to 40% by weight) did not have an appreciable effect on the duration of release over the 42 days, but, did alter the amount of initial release from the formulation.

TABLE 3
Salt-free cetrorelix PLGA formulation compositions

| | | | | | | Theo. | | |
|---|---|---|---|---|---|---|---|---|
| | | | | | | Cetrorelix | PLGA | Total |
| | Cetrorelix | RG502H | RG752H | NMP | Total | Loading | Solution | Solid |
| ID | (mg) | (mg) | (mg) | (mg) | (mg) | (%, w/w) | (%, w/w)* | (%, w/w) |
| VH-022-001 | 365.5 | 677.22 | 0.0 | 2688 | 3730.4 | 9.8 | 20.1% | 28.0 |
| VH-023-001 | 291.3 | 408.28 | 405.7 | 1885 | 2990.5 | 9.7 | 30.2% | 37.0 |
| VH-024-001 | 291.5 | 0.0 | 1084.6 | 1618 | 2993.6 | 9.7 | 40.1% | 46.0 |

*PLGA (%, w/w) = PLGA wt ÷ (PLGA wt + Total Solvent wt)

TABLE 4
Salt-free cetrorelix PLGA formulation rat pharmacokinetic results

| | Cₘₐₓ | Tₘₐₓ | AUC_{0-42 day} | C_{ave, 42 day} | Cₘₐₓ/ | AUC_{0-24 hr} | AUC_{0-24 hr}/ |
|---|---|---|---|---|---|---|---|
| ID | (ng/mL) | (hr) | (ng/mL * hr) | (ng/mL) | C_{ave, 42 day} | (ng/mL * hr) | AUC_{0-42 day} |
| VH-022-001 | 283.7 | 1 | 16272.7 | 16.1 | 17.6 | 2761.9 | 17.0% |
| VH-023-001 | 218.7 | 1 | 18270.5 | 18.1 | 12.1 | 2206.9 | 12.1% |
| VH-024-001 | 218.7 | 1 | 16406.1 | 16.3 | 13.4 | 2191.0 | 13.4% |

### Example 5

### Salt Compositions of Cetrorelix

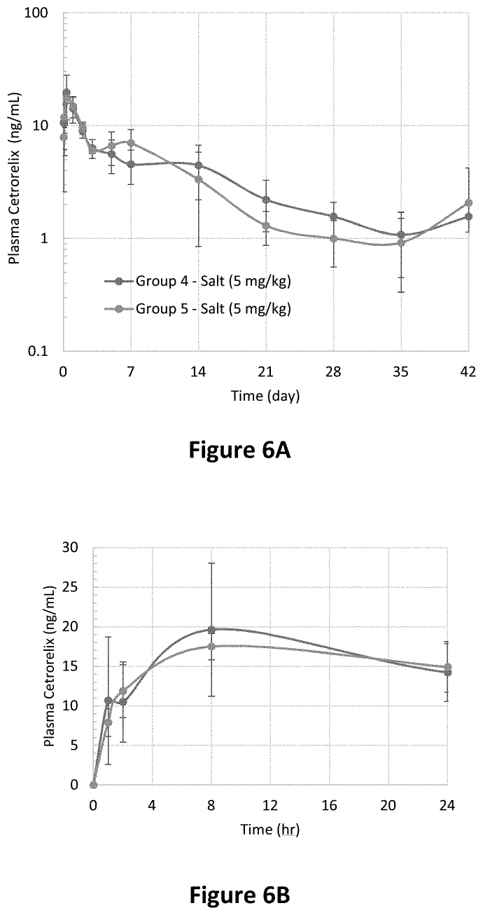

The pharmacokinetics of cetrorelix formulations of either Ca Pamoate or Na Oleate salt were tested in rats through subcutaneously implanting a single 5 mg/kg salt suspension in a vehicle solution (20 mM K-Phos Buffer, 2.5% Mannitol, 3.5% CMC, 0.1% PS80) and monitoring cetrorelix levels in plasma over 6 weeks. The results are summarized in Table 5 and FIGS. 6A and 6B. All formulations showed detectable plasma cetrorelix for greater than 42 days. Cₘₐₓ/C_{ave,42day} varied from 2.4 to 3.1 and the percentage of day 1 release related to 42 days about 11%.

TABLE 5
Salt-containing cetrorelix formulations rat pharmacokinetic results.

| | Cₘₐₓ | Tₘₐₓ | AUC_{0-42 day} | C_{ave, 42 day} | Cₘₐₓ/ | AUC_{0-24 hr} | AUC_{0-24 hr}/ |
|---|---|---|---|---|---|---|---|
| ID | (ng/mL) | (hr) | (ng/mL * hr) | (ng/mL) | C_{ave, 42 day} | (ng/mL * hr) | AUC_{0-42 day} |
| Group 4 - | 19.6 | 8 | 3444.0 | 3.4 | 3.1 | 376.9 | 10.9% |
| Salt | | | | | | | |
| Ca Pamoate | | | | | | | |
| Group 5 - | 17.5 | 8 | 3331.9 | 3.3 | 2.4 | 361.1 | 10.8% |
| Salt | | | | | | | |
| Na Oleate | | | | | | | |

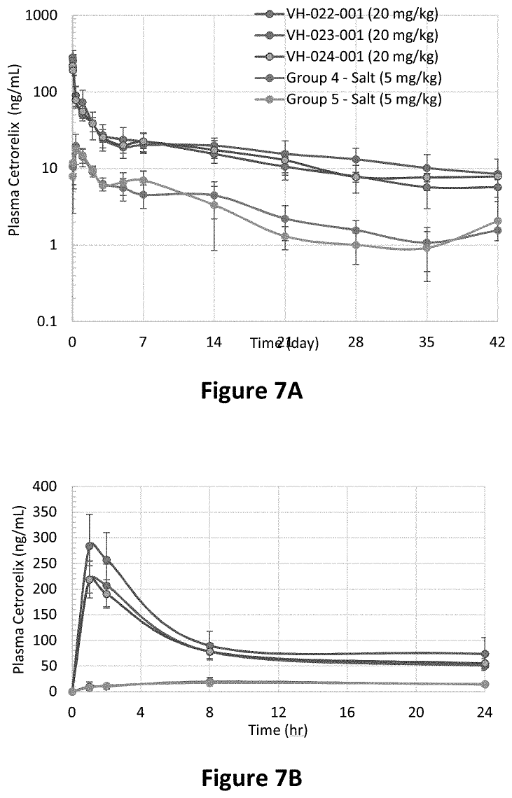

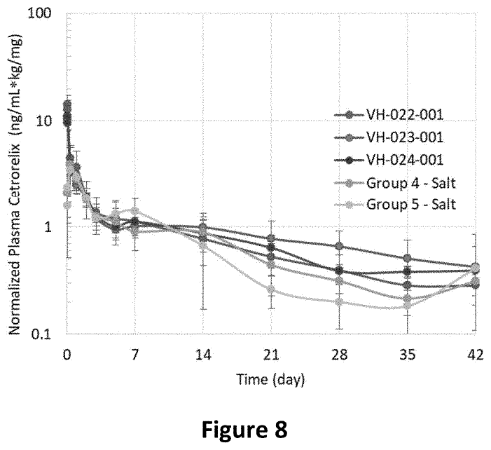

There was no principal difference between pharmacokinetics of the two cetrorelix salt compositions in the initial 24 hour initial release (FIG. 6B) and over the long term (FIG. 6A). Moreover, while salt compositions of cetrorelix resulted in lower plasma cetrorelix compared to the salt free formulations in the long term (FIG. 7A), this difference could be attributable to the different dose (FIG. 8). However, dose difference did not account for the far greater peak concentration observed during the initial release phase with cetrorelix in PLGA formulations. Indeed, the salt composition all but eliminated the initial release peak that was observed in PLGA formulations (FIG. 7B).

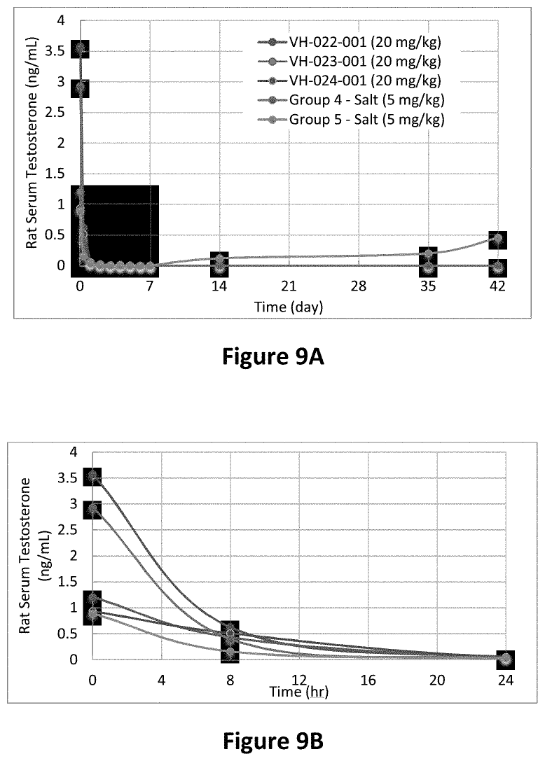

The downstream physiological effects of cetrorelix microspheres administration were assessed through monitoring testosterone levels in rats. All the formulations caused notable drop in serum testosterone levels after 24 hours (FIG. 9B). These low levels were maintained for the entire 6 weeks monitoring period in all rats except those treated with Na Oleate salt compositions (FIG. 9A).

The total amount of cetrorelix released was assessed using the method of Schwahn et al., Drug Metabolism & Disposition, Vol. 28, No. 1, p 10, incorporated by reference, assuming rat weight ranging from 250 to 400 g, 10 mg/kg dose, and AUC (ng/mL*hr)=618.1.

Based on these assumptions, AUC for 20 mg/kg dose (ng/mL*hr) was assumed to be 123,620 and AUC for 5 mg/kg dose (ng/mL*hr) was assumed to be 30,905. The calculations based on the above assumptions resulted in estimated percentage of cetrorelix released (up to 42 days) ranging from 11 to 15% of the amount initially present in the microspheres. (See Table 6). These results clearly show that the salt compositions have a lower initial release compared to cetrorelix in PLGA alone.

TABLE 6
Summary of pharmacokinetic data for cetrorelix salt and PLGA formulations.

| | | | | | | Est. % Cetrorelix |
|---|---|---|---|---|---|---|
| | | Dose | Cₘₐₓ | Tₘₐₓ | AUC_{0-42 day} | Released |
| ID | PLGA | (mg/kg) | (ng/mL) | (hr) | (ng/mL * hr) | (up to 42 days) |
| VH-022-001 | RG502H, 20% | 20 | 283.7 | 1 | 16272.7 | 13.2 |
| VH-023-001 | RG502H/RG752H, 30% | 20 | 218.7 | 1 | 18270.5 | 14.8 |
| VH-024-001 | RG752H, 40% | 20 | 218.7 | 1 | 16406.1 | 13.3 |
| Group 4 - Salt | Ca Pamoate | 5 | 19.6 | 8 | 3444.0 | 11.1 |
| Group 5 - Salt | Na Oleate | 5 | 17.5 | 8 | 3331.9 | 10.8 |

### Example 6

### In Vitro Cetrorelix Release Studies of Cetrorelix Salt Compositions in PLGA Formulations

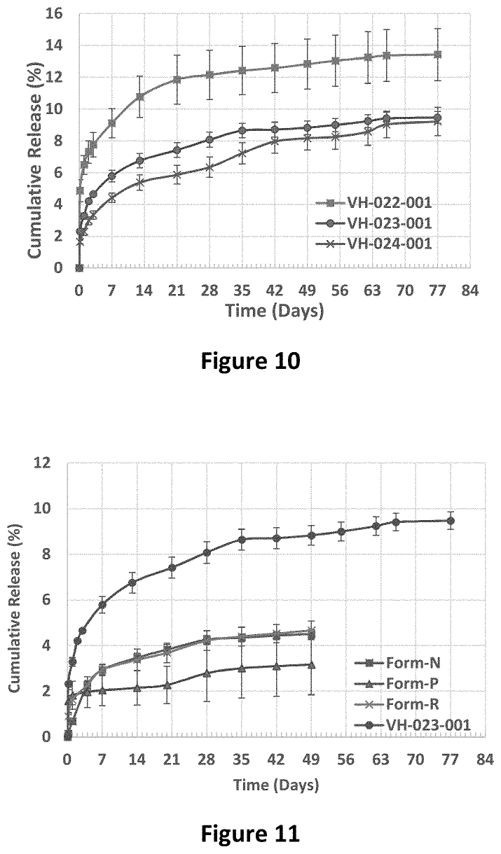

To supplement the in vivo pharmacokinetic data and further optimize the compositions of cetrorelix in PLGA, in vitro release studies were carried out wherein 45 mg of cetrorelix were incubated in 0.5 ml Tris Mannitol, pH 7.4 at 37° C. The results for in vitro release for the salt-free formulations used in pharmacokinetic studies are summarized in FIG. 10. In contrast to the in vivo data, the increase of PLGA content in the composition from 20% to 30% resulted in marked reduction of cetrorelix release rate and of cumulative release. Surprisingly, further increase of PLGA content from 30% to 40%, while resulting in further decrease in release rate, also yielded negligible change in cumulative release levels, especially in the long term. Moreover, in all formulations the cumulative release appears to plateau after about 60 days suggesting that the maximum cumulative release has been achieved.

In order to further explore the effect of salt composition on cetrorelix release, several salt compositions of cetrorelix were prepared as formulations in 30% total PLGA solution (1:1 ratio of RG502H (50:50 lactide:glycolide) and RG752H (75:25 lactide:glycolide)) (Table 7), then tested and compared with salt-free PLGA formulations.

TABLE 7
Salt-containing RG502H/RG752H (30% PLGA) cetrorelix formulations

| | | Cetrorelix | Cetrorelix | | | | Cetrorelix | PLGA | |
|---|---|---|---|---|---|---|---|---|---|
| | | Salt | Content | PLGA* | NMP | Total | Loading | Solution | Total Solid |
| ID | Salt | (mg) | (%, w/w) | (mg) | (mg) | (mg) | (%, w/w) | (%, w/w) | (%, w/w) |
| Form-N | Ca Pamoate | 63.29 | 77% | 135.0 | 316.3 | 514.6 | 9.5 | 29.9 | 38.5 |
| Form-P | Na Oleate | 58.80 | 83% | 134.7 | 317.4 | 510.9 | 9.6 | 29.8 | 37.9 |
| Form-R | Ca Citrate | 62.20 | 78% | 134.9 | 316.5 | 513.6 | 9.4 | 29.9 | 38.4 |

*PLGA = 50:50 RG752H:RG502H (w:w)

The results show that the presence of salt markedly decreases cumulative cetrorelix release levels (FIG. 11). While Na Oleate was particularly effective, all salts resulted in lower cumulative release compared to the corresponding salt free composition. In addition, cetrorelix cumulative release in the case of salt compositions appeared to reach a plateau after 49 days. At the same time Na Oleate and Ca Citrate did not appear to have any effect on the initial release rate. The presence of Ca Pamoate decreased the initial release rate.

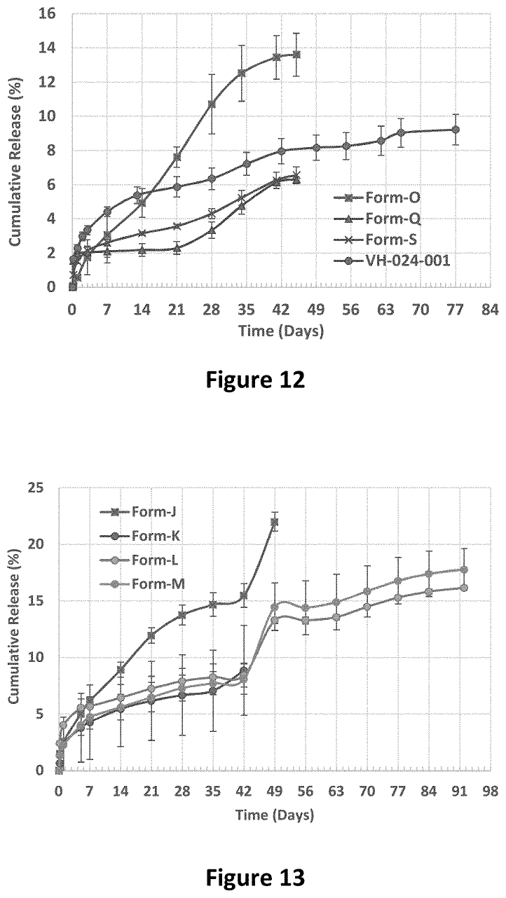

Surprisingly, when the polymer content was increased to 40% and PLGA was RG752H (75:25 lactide:glycolide) (Table 8), the presence of Ca Pamoate slowed the initial release rate but dramatically (nearly 3-fold) increased cumulative cetrorelix release, as compared with salt free formulation. On the other hand, similarly to the RG502H/RG752H data, Na Oleate and Ca Citrate did not appear to have any effect on the initial release rate although these formulations displayed a modest increase in cumulative release levels (FIG. 12).

TABLE 8
Salt-containing RG752H (40% PLGA) cetrorelix formulations

| | | Cetrorelix | Cetrorelix | | | | Cetrorelix | PLGA | Total |
|---|---|---|---|---|---|---|---|---|---|
| | | Salt | Content | RG752H | NMP | Total | Loading | Solution | Solid |
| ID | Salt | (mg) | (%, w/w) | (mg) | (mg) | (mg) | (%, w/w) | (%, w/w) | (%, w/w) |
| Form-O | Ca Pamoate | 62.95 | 77% | 180.5 | 268.4 | 511.8 | 9.5 | 40.2 | 47.6 |
| Form-Q | Na Oleate | 58.53 | 83% | 180.6 | 269.4 | 508.6 | 9.6 | 40.1 | 47.0 |
| Form-S | Ca Citrate | 56.88 | 78% | 166.2 | 246.5 | 469.5 | 9.4 | 40.3 | 47.5 |

To investigate the effect of polymer and N-methyl-2-pyrrolidone (NMP) on cetrorelix release rate and cumulative release, cetrorelix salt compositions were prepared in formulations using 40% total PLGA (1:1 RG502H and RG752H) or 40% RG752S (ester end capped), with NMP or DMSO as polymer solvent (Table 9) were tested.

TABLE 9
Cetrorelix formulations with different polymers and solvent at 40% total PLGA.

| | | | RG502H + | | | | Cetrorelix | PLGA | Total |
|---|---|---|---|---|---|---|---|---|---|
| | Cetrorelix | RG752S | RG752H | NMP | DMSO | Total | Loading | Solution | Solid |
| ID | (mg) | (mg) | (mg)* | (mg) | (mg) | (mg) | (%, w/w) | (%, w/w)** | (%, w/w) |
| Form-J | 45.3 | 167.43 | 0 | 257 | 0 | 470.1 | 9.6 | 39.4 | 45.3 |
| Form-K | 45.4 | 168.55 | 0 | 0 | 251 | 465.2 | 9.8 | 40.1 | 46.0 |
| Form-L | 45.6 | 0 | 166.98 | 252 | 0 | 464.2 | 9.8 | 39.9 | 45.8 |
| Form-M | 45.9 | 0 | 169.87 | 0 | 252 | 467.3 | 9.8 | 40.3 | 46.2 |

*RG502H:RG752H (w:w) = 50:50
**PLGA (%, w/w) = PLGA wt ÷ (PLGA wt + Total Solvent wt)

The results are summarized in FIG. 13 and Table 10. Surprisingly, the NMP formulation of cetrorelix in 40% RG752S resulted in a dramatic increase in both cetrorelix release rate and total release as compared to the same formulation in DMSO solution. Interestingly, the mixed polymer formulation in 40% total PLGA (1:1 mixture of RG502H and RG752H) displayed NMP-independent multiphasic cetrorelix release profile consisting of initial burst phase followed by a first plateau phase at about 8% cumulative release, achieved on day 42, followed by the second release phase on days 42-49, and subsequently followed by a second plateau phase, at approximately 17% cumulative release, achieved on day 92. The total recovery from the formulation composition in the in vitro release experiment is about 100% indicating that despite the seemingly limited cumulative release for all formulations (<30%), the cetrorelix remains intact within the depot formulations.

TABLE 10
Mass balance (total recovery) from in vitro release data
for 40% RG752S formulations in either NMP or DMSO

| ID | Total Release (%) | Residual Loading (%) | Total Recovery (%) |
|---|---|---|---|
| Form-J | 22.0 (743.3 μg) | 89.0 | 111.0 |
| Form-K | 11.1 (362.0 μg) | 89.4 | 100.2 |

### Example 7

### Cetrorelix Salt Compositions and Cetrorelix PLGA Formulations Pharmacokinetics in Rats

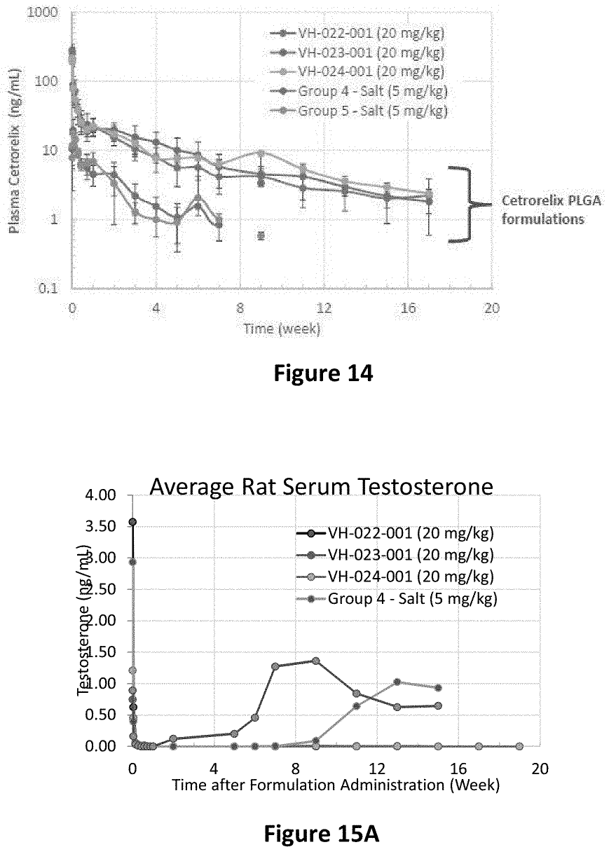

Several salt-free cetrorelix-loaded gel formulations having different polymer contents were used for PK Study (see Table 6 for PLGA content of salt-free cetrorelix-loaded gel formulations). The salt-free preparations (<1 ml) were subcutaneously implanted into rats at a single 20 mg/kg dose and cetrorelix levels in plasma were monitored over 17 weeks (119 days). The results are summarized in Table 11 and FIG. 14. All formulations showed detectable plasma cetrorelix ≥119 days. The percentage of day 1 release related to 119 days ranged from 9 to 13%. Importantly, the amount of polymer in the preparation did not seem to have any statistically significant effect on cetrorelix release.

TABLE 11
Rat study results up to 119 days: Pharmacokinetic data of
salt-free and salt-containing cetrorelix gel formulations

| | Cetrorelix | Cₘₐₓ | Tₘₐₓ | AUC_{0-119 day} | AUC_{0-24 hr} | AUC_{0-24 hr}/ |
|---|---|---|---|---|---|---|
| ID | Dose (mg/kg) | (ng/mL) | (hr) | (ng/mL * hr) | (ng/mL * hr) | AUC_{0-119 day} |
| VH-022-001 | 20 | 283.7 | 1 | 22,088.05 | 2761.9 | 12.5% |
| Salt-free | | | | | | |
| VH-023-001 | 20 | 218.7 | 1 | 25,418.35 | 2206.9 | 8.7% |
| Salt-free | | | | | | |
| VH-024-001 | 20 | 218.7 | 1 | 26,173.31 | 2191.0 | 8.4% |
| Salt-free | | | | | | |
| Group 4 - Salt | 5 | 19.6 | 8 | 4,359.27* | 376.9 | 8.6%* |
| Ca Pamoate | | | | | | |
| Group 5 - Salt | 5 | 17.5 | 8 | 3,854.37* | 361.1 | 9.4%* |
| Na Oleate | | | | | | |

*Groups 4 and 5 had no plasma collected after 91 days.

In addition, pharmacokinetics of cetrorelix gel formulations containing either Ca Pamoate or Na Oleate salt were tested in rats through subcutaneously implanting a single 5 mg/kg gel dose in a vehicle solution (20 mM K-Phos Buffer, 2.5% Mannitol, 3.5% CMC, 0.1% PS80) and monitoring cetrorelix levels in plasma for 91 days. No plasma was collected after day 91 for rats into which cetrorelix gel formulations containing either Ca Pamoate or Na Oleate salt were implanted. (See Table 7 for salt-containing RG502H/RG752H (30% PLGA) cetrorelix gel formulations). The results are summarized in Table 11 and FIG. 14. All formulations showed detectable plasma cetrorelix ≥over 7 weeks (≥49 days). The percentage of day 1 release related to 91 days was about 9%.

There was no principal difference between pharmacokinetics of cetrorelix gel formulations containing Ca Pamoate and those containing Na Oleate both in the initial 24 hour burst release phase (FIG. 14) and over the long term (FIG. 14). Although, salt-containing cetrorelix gel resulted in lower plasma cetrorelix compared to the salt free formulations in the long term (FIG. 14), this difference could be attributable to the different dose (FIG. 14 and Table 11). However, dose difference did not account for the far greater peak concentration observed at the initial burst release phase when salt free formulations were used. Indeed, addition of salt all but eliminated the sharp peak observed in its absence (FIG. 14).

The downstream physiological effects of cetrorelix gel administration were assessed through monitoring testosterone levels in rats. All the formulations caused notable drop in serum testosterone levels after 24 hours (FIG. 15B). These low levels were maintained for the entire 19 weeks monitoring period in all rats except those treated with Na Oleate-containing microspheres, whose testosterone levels began to increase after week 5 (FIG. 15A). In contrast, the testosterone levels of rats treated with the Ca-Oleate began to increase after week 9 (FIG. 15A).

The total amount of cetrorelix released was assessed using the method of Schwahn et al., Drug Metabolism & Disposition, Vol. 28, No. 1, p 10, assuming rat weight ranging from 250 to 400 g, 10 mg/kg dose, and AUC (ng/mL*hr)=618.1.

Based on these assumptions, AUC for 20 mg/kg dose (ng/mL*hr) was assumed to be 123,620 and AUC for 5 mg/kg dose (ng/mL*hr) was assumed to be 30,905. The calculations based on the above assumptions results in estimated percentage of cetrorelix released (up to 119 days) ranging from 9 to 13% of the amount initially present in the gel. (See Table 11).

### Example 8

### Enhancing the Level of Cetrorelix

To further explore the effect of polymer on cetrorelix release, several formulations of cetrorelix in PLGA (Table 13) were tested and compared within a second rat study and in dogs. Variables were PLGA polymer type, peptide source, solvent, drug load, and % PLGA in solution.

TABLE 13
Cetrorelix PLGA formulation compositions

| | | | | Cetrorelix | PLGA |
|---|---|---|---|---|---|
| | | | | Loading | Solution |
| | | PLGA Type | | (%, | (%, |
| ID | API | Solvent | Solvent | w/w) | w/w) |
| VH-023-003 | CPC | RG502H/ | NMP | 10.2 | 30.1% |
| | | RG752H | | | |
| VH-030-001 | Hemmo | RG752H | NMP | 10.2 | 29.9% |
| VH-030-002 | CPC | RG752H | NMP | 10.1 | 29.9% |
| VH-031-001 | CPC | RG752H | NMP | 9.9 | 25.1% |
| VH-032-001 | CPC | RG752H | NMP | 10.0 | 20.0% |
| VH-033-001 | CPC | R202H | NMP | 10.6 | 30.0% |
| VH-034-001 | CPC | RG752H | NMP | 12.3 | 30.1% |
| VH-035-001 | CPC | RG752H | DMSO | 10.1 | 30.0% |

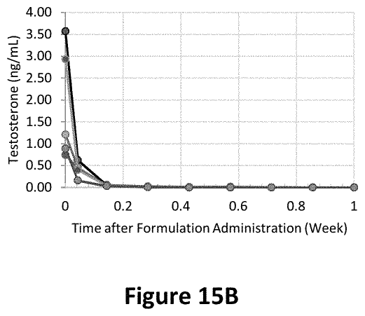

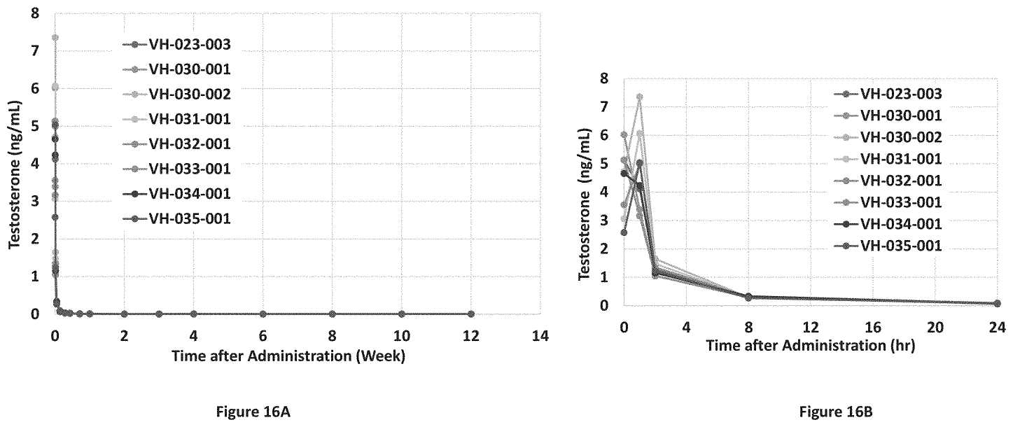

In a second rat study serum testosterone and cetrorelix levels were measured over 12 weeks. FIG. 16A shows the testosterone level in rats at 12 weeks. The formulations found within Table 13 were injected in rats and then the testosterone was measured at 0, 1, 2, 8 and 24 hours and then daily until the end of the first week and then biweekly until week 4 and then weekly thereafter. As shown in FIG. 16B, the level of testosterone dropped below 1 ng/ml within 8 hours and the level was maintained over 12 weeks with no formulations showing break through (elevated testosterone).

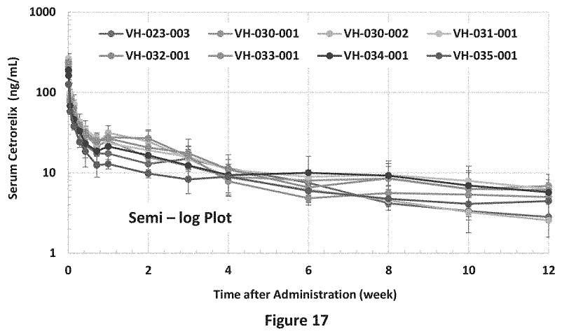

FIG. 17 shows the cetrorelix levels in the 2^{nd} rat study over the same 12-week period. The levels of cetrorelix were all above 2 ng/ml which is the level required to inhibit testosterone production.

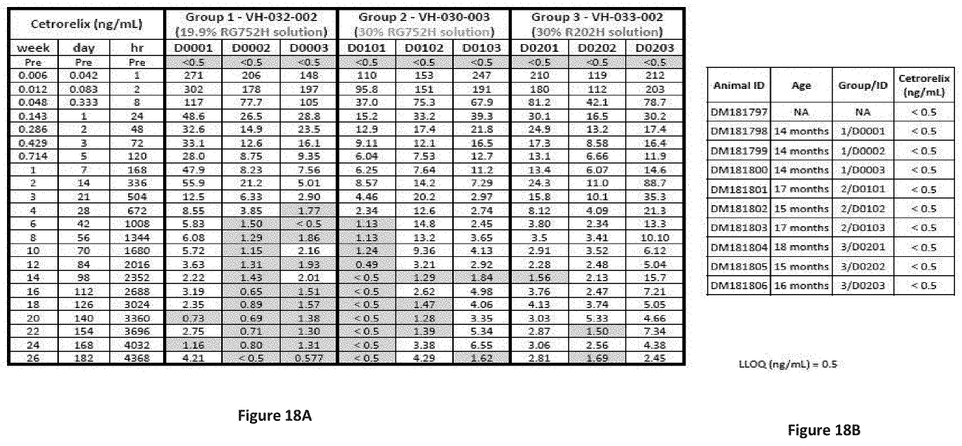

Three PLGA cetrorelix formulations were selected for further testing of cetrorelix pharmacokinetics in dogs. Three cetrorelix formulations that consisted of different PLGA/NMP solutions were used: 20% RG752H (VH-032-002), 30% RG752H (VH-030-003), and 30% R202H (VH-033-002). After the initial dose, the animals serum concentration of cetrorelix was measured for 26 weeks. In all three groups at least one animal still had a serum concentration above 2 ng/ml at 26 weeks as shown in FIG. 18A. FIG. 18B shows the background levels of cetrorelix within the studies animals one week prior to dosing, which was less than 0.5 ng/ml for all the animals.

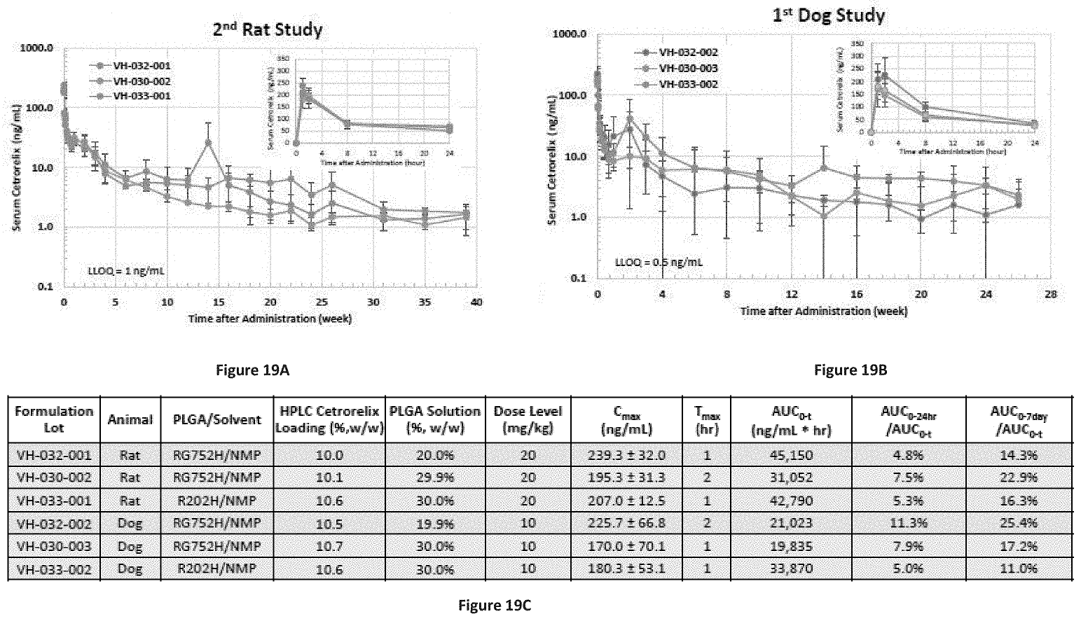

FIG. 19 compares the cetrorelix data from the 1^{st} dog trials to the 2^{nd} rat trial up to 39 weeks. All three formulations injected into the rats had quantifiable levels of cetrorelix after 39 weeks as shown in FIG. 19A. Similarly, FIG. 19B shows that the three formulations injected into the dogs in the first trial had quantifiable levels of cetrorelix after 26 weeks. The pharmacokinetic data is tabulated in FIG. 19C.

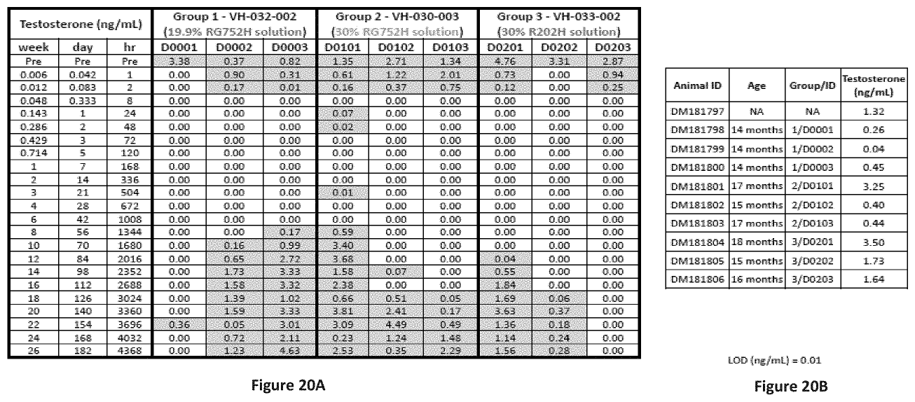

Serum testosterone data was collected in the 1^{st} dog trial to demonstrate efficacy of the cetrorelix in the formulation. FIG. 20A shows the levels of testosterone the dogs had within each group. All the dogs had quantifiable levels of testosterone one week prior to injection, as shown in FIG. 20B, but after the injection the animals only had a sustained quantifiable level of testosterone starting at week 8 and some of the animals did not have a quantifiable level of testosterone even after 26 weeks.

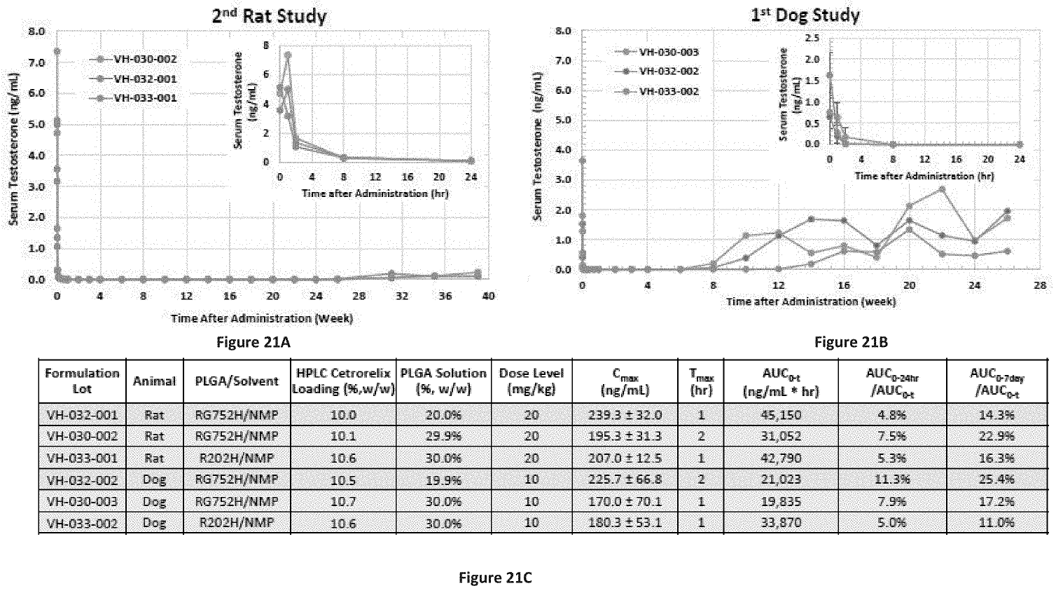

FIG. 21 compares the data from the 2^{nd} rat and 1^{st} dog study results for testosterone levels. FIG. 21A shows that the rats did not have a resurgence of testosterone for any of the formulations until week 26. Whereas FIG. 21B shows that the dogs started to regain testosterone after week 6, but it did take several weeks for the animals to regain the levels they had before the cetrorelix injection. The data is tabulated in FIG. 20A.

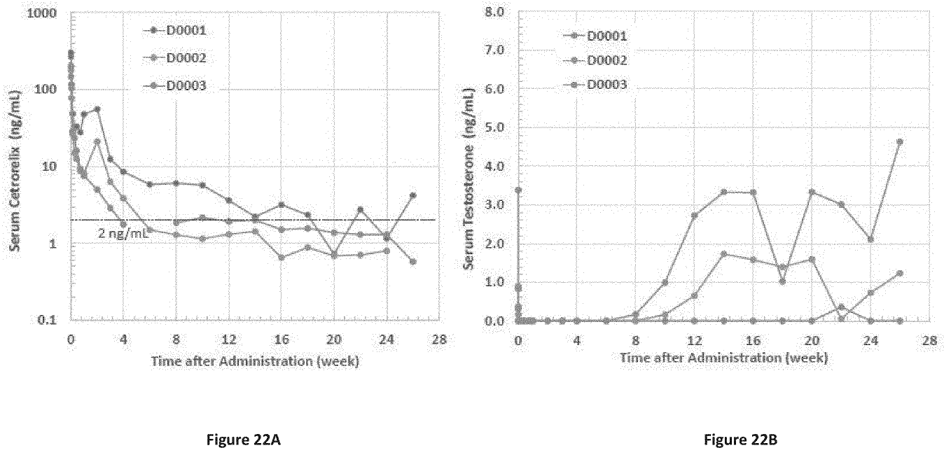

FIG. 22A shows the level of cetrorelix for the 19.9% RG752H solution formulation in the 1st dog trial. The serum level of cetrorelix stayed above 2 ng/ml until at least week 4 and for one animal up to week 14. FIG. 22B shows the corresponding measured level of serum testosterone for the same animals. As long as the cetrorelix level was above 2 ng/ml, the testosterone level was below the quantifiable limit for all three animals. Once cetrorelix level drops below 2 ng/ml the testosterone level increased.

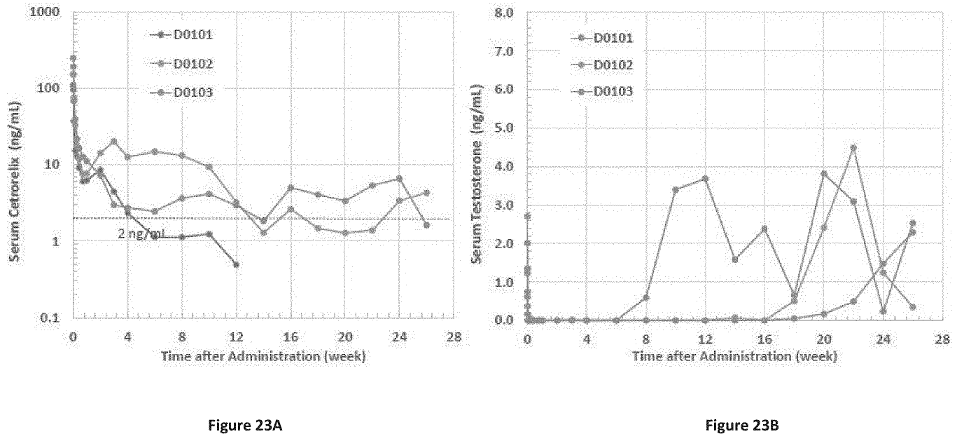

FIG. 23A shows the level of cetrorelix for the 30% RG752H solution formulation in the 1^{st} dog trial. The serum level of cetrorelix stayed equal to or greater than 2 ng/ml until at least week 4 and for one animal until week 14. FIG. 23B shows the corresponding measured level of serum testosterone for the same animals. As long as the cetrorelix level was above 2 ng/ml the testosterone level was below the quantifiable limit for all three animals. Once cetrorelix level drops below 2 ng/ml the testosterone level increased.

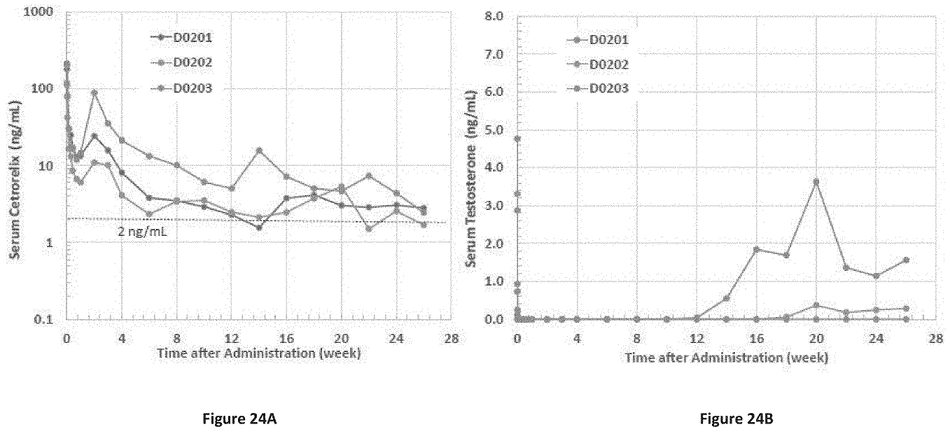

FIG. 24A shows the level of cetrorelix for the 30% R202H solution formulation in the 1^{st} dog trial. The serum level of cetrorelix stayed above 2 ng/ml until at least week 13 and for one animal even after 26 week the level still was above 2 ng/ml. FIG. 24B shows the corresponding measured level of serum testosterone for the same animals. As long as the cetrorelix level was above 2 ng/ml the testosterone level was below the quantifiable limit for all three animals. Once cetrorelix level drops below 2 ng/ml the testosterone level increased. Therefore, the animal whose level of cetrorelix did not dip below 2 ng/ml did not experience an increase in testosterone even after 26 weeks.

In sum, the results in FIGS. 16-24 demonstrate that testosterone level goes up only once cetrorelix level drops less than 2 ng/ml.

### Example 9

### Cetrorelix Deposit

A subject is injected with a depot injection, usually subcutaneously that deposits the drug in a localized mass that is gradually absorbed by the surrounding tissue. The depot injection has a consistent release of the drug over a sustained period.

Cetrorelix, in any of the formulations described herein, is injected subcutaneously as a depot, and the drug releases over at least 90 days. There is an immediate suppression of testosterone, without a concurrent surge upon the initial or repeated injection of the formulation of cetrorelix.

### Example 10

### Prostate Cancer Treatment

In a clinical trial done with 50 adult-male, prostate cancer subjects in an open label study. The subjects will have a histologically confirmed diagnosis of adenocarcinoma of the prostate. The subjects are stratified into 5 different categories based on disease progression with each group having ten patients. The groups will all be injected once with 0.375 g of the 45 mg formulation subcutaneously.

The subjects will have their blood taken every week to have the serum levels of cetrorelix and testosterone analyzed. Every 3 weeks, biopsies are performed to ascertain the size of the adenocarcinoma of the prostate. It is assumed that as long as the level of cetrorelix is above 2 ng/ml the levels of testosterone within the subject would be extremely low, which will cause the adenocarcinoma to shrink and the tumor to go into remission.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for decreasing the level of testosterone in a subject in a cetrorelix treatment comprising administering to the subject a composition of multi-block copolymer, a biocompatible polar aprotic solvent, and cetrorelix, wherein the level of cetrorelix is maintained at more than 2 ng/mL in the subject.

2. The method according to claim 1, wherein the composition releases cetrorelix for three, six, nine, or twelve months.

3. The method according to claim 2, wherein the multi-block copolymer has at least one of poly-lactic, co-glycolic acid, poly-lactic acid, poly-glycolic acid, polyethylene glycol, poly (3-hydroxybutyrate), or polycaprolactone.

4. The method according to claim 3, wherein the multi-block copolymer has poly-lactic and co-glycolic acid in a molar ratio between about 1:1 to about 6:1 poly-lactic acid:poly-glycolic acid.

5. The method according to claim 1, wherein the biocompatible polar aprotic solvent is at least one of N-methyl-2-pyrrolidone, 2-pyrrolidone, N,N-dimethylformamide, N,N-dimethylacetamide, dimethyl sulfoxide, propylene carbonate, caprolactam, triacetin, benzyl benzoate, or propylene glycol.

6. The method according to claim 5, wherein the biocompatible polar aprotic solvent is N-methyl-2-pyrrolidone.

7. The method according to claim 1, wherein the cetrorelix is present in an amount of at least 5% of the weight of the composition.

8. The method according to claim 1, wherein composition has an area under the curve between 10000 ng/ml*hr and 30000 ng/ml*hr.

9. The method according to claim 1, wherein the composition is in the form of a microsphere, hydrogel, or flowable composition.

10. The method according to claim 1, wherein the composition is a hydrogel.

11. The method according to claim 10, wherein the composition is biodegradable.

12. The method according to claim 1, wherein the cetrorelix is present in an amount of about 50 mg to about 300 mg.

13. The method according to claim 1, wherein the cetrorelix treatment treats a disease associated with gonadotropin-releasing hormone (GnRH).

14. The method according to claim 13, wherein the cetrorelix treatment comprises suppression of testosterone, FSH, and LH production for the treatment of prostate cancer and benign prostatic hyperplasia, directly blocking GnRH receptors on prostate cells for treatment of prostate cancer and benign prostatic hyperplasia, controlled ovarian stimulation for assisted reproductive techniques, treatment of uterine myoma, suppression of ovarian function while undergoing chemotherapy, treatment of breast cancer, treatment of ovarian cancer, male contraception, and female contraception.
